(19) United States
(12) Patent Application Publication
(10) Pub. No.: US 2010/0199362 A1
(43) Pub. Date: Aug. 5, 2010

(54) WNT LIGANDS INVOLVED IN BLOOD-BRAIN BARRIER DEVELOPMENT AND USES THEREFOR

(75) Inventors: Andrew McMahon, Lexington, MA (US); Jan Stenman, Cambridge, MA (US)

Correspondence Address: FOLEY HOAG, LLP (w/HUV HMV);PATENT GROUP, 155 SEAPORT BLVD., BOSTON, MA, 02210-2600, US

(73) Assignee: President and Fellows of Harvard College, Cambridge, MA (US)

(21) Appl. No.: 12/597,669

(22) PCT Filed: Apr. 28, 2008
(86) PCT No.: PCT/US08/61768

§ 371 Date: Apr. 12, 2010

Related U.S. Application Data
(60) Provisional application No. 60/926,523, filed on Apr. 26, 2007.

(51) Int. Cl. A01K 67/00; G01N 33/53; A61K 38/02; A61K 9/127; A61K 39/395; A61K 31/7105; A61K 38/16; A61K 51/00; A61P 25/00

(52) U.S. Cl. 800/9; 435/7.1; 514/2; 424/450; 424/130.1; 514/44 A; 514/12; 424/1.11

(57) ABSTRACT

Provided are methods of diagnosing and treating vascular disorders of the CNS or disorders of the blood-brain barrier comprising the use of Wnt ligands involved in the canonical Wnt signaling pathway.

### RELATED APPLICATION

This application claims the benefit of priority to Provisional Application 60/926,523, filed Apr. 26, 2007, which is hereby incorporated by reference in its entirety.

### BACKGROUND

The development of the central nervous system (CNS) vasculature is believed to occur in three steps: vasculogenesis, angiogenesis, and barriergenesis. In the mouse, paraxial mesoderm-derived angioblasts give rise to the perineural vascular plexus (PNVP) surrounding the developing CNS, in a process that critically depends upon vascular endothelial growth factor (VEGF) produced by the neuroepithelium. At the spinal cord level, some endothelial cells in the PNVP start sending sprouts into the CNS at E9.5 ultimately leading to the formation of an intraneural vascular plexus (INVP). The development of the mature BBB occurs over a prolonged period of time, but phenotypic changes in CNS endothelial cells occur very early, with expression of Glut1 preceding the appearance of the first sprouts, and lyn and P-Glycoprotein shortly thereafter. Several signaling pathways that control early development of the mouse vascular system, e.g., the VEGF/Flk1 and Ang1/Tie2 signaling pathways, are also critical for the development of the CNS vasculature. As VEGF has been demonstrated to be expressed already as early as E8.5 in the CNS and control PNVP formation, it appears likely that while VEGF most certainly remains critical for normal CNS vascularization, other signaling pathways are required to determine the exact timing of CNS vascular invasion and to endow the CNS vasculature with its unique properties.

Diseases of the brain's vasculature cause more deaths than circulatory disorders of any other organ except the heart. They are the third leading cause of death after heart disease and cancer. Brain cells die extremely rapidly when deprived of their blood supply and are hard to replace. Survivors are oftentimes severely incapacitated. Additionally, brain and other CNS tumors require a blood supply to meet their increased metabolic demands, and therefore induce the development of tumor-associated vasculature. Such vasculature is pathophysiological in its cellular architecture, leading to cerebral edema. The identification of genes that influence the development of the CNS vasculature and blood-brain barrier may provide useful therapeutic targets for treating vascular disorders of the CNS and disorders of the blood-brain barrier. Further, there remains an unmet medical need for new and effective therapeutic agents to treat vascular disorders of the CNS and disorders of the blood-brain barrier.

### SUMMARY

The present invention is based, at least in part, on our discovery that Wnt ligands Wnt7a and Wnt7b produced by the neuroepithelium act directly on the central nervous system blood vessels and blood vessel precursors via the canonical Wnt signaling pathway to both control CNS vascularization and establish properties of the BBB.

In one aspect, the invention is directed to a method for identifying an agent that modulates Wnt ligand activity or canonical Wnt signaling pathway component activity, by contacting a Wnt ligand or canonical Wnt signaling pathway component, or a portion thereof that is sufficient to promote canonical Wnt signaling, with a test agent, and determining the level of activity from the Wnt ligand or canonical Wnt signaling pathway component or portion thereof in the presence of the test agent relative to the absence of the test agent, wherein a change in activity indicates that the test agent is an agent that modulates the activity of the Wnt ligand or canonical Wnt signaling pathway component. In some embodiments, the test agent stimulates the level of activity of the Wnt ligand or canonical Wnt signaling pathway component or portion thereof relative to the absence of the test agent. Alternatively, the test agent inhibits the level of activity of the Wnt ligand or portion thereof relative to the absence of the test agent. The Wnt ligand is a human or mouse Wnt7a or Wnt7b. In certain embodiments, the Wnt ligand or canonical Wnt signaling pathway component is present in an animal model and the Wnt ligand activity or canonical Wnt signaling pathway component activity is assessed by measuring whether the canonical Wnt pathway is modulated in the animal model. Alternatively, the Wnt ligand or canonical Wnt signaling pathway component is present in a cell and the Wnt ligand activity or canonical Wnt signaling pathway component activity is assessed by measuring whether the canonical Wnt pathway is modulated in the cell. A test agent is preferably a molecule identified in a library of molecules, such as a small molecule identified from a small molecule library. The invention also provides a pharmaceutical composition containing an agent identified by the methods described herein.

In another aspect the invention provides an animal model for vascular disorders of the CNS and disorders of the blood-brain barrier, that includes an animal having a mutation in the gene encoding a Wnt ligand or canonical Wnt signaling pathway component, which mutation prevents the canonical Wnt signaling pathway from functioning normally and causes a hemorrhaging phenotype. The mutation preferably includes a Wnt7a/b double mutant, and the animal is preferably a mouse.

In a further aspect, the invention provides a method for treating a vascular disorder of the CNS or disorder of the blood-brain barrier in a subject, by administering to the subject an effective amount of a modulator of Wnt ligand activity or canonical Wnt signaling pathway component activity, wherein the modulator of Wnt ligand activity or canonical Wnt signaling pathway component activity normalizes the activity of the canonical Wnt signaling pathway, thereby treating the vascular disorder of the CNS or disorder of the blood-brain barrier in the subject. The Wnt ligand is preferably a mouse or human Wnt7a or Wnt7b.

The invention provides in another aspect a method of modulating blood-brain barrier (BBB) permeability by administering an agent to a subject, wherein said agent targets Wnt7a or Wnt7b that is present in the brain. In certain embodiments, the agent increases BBB permeability. Alternatively, the agent decreases BBB permeability. The modulation of BBB permeability may be reversible, or irreversible. The agent is an inorganic molecule, peptide, peptide mimetic, antibody, liposome, small interfering RNA, antisense, aptamer, or external guide sequence, or a combination thereof. In one embodiment, the agent is a secreted Wnt 7a/7b signaling inhibitor.

In another aspect, the invention provides a method of treating a disease or disorder of the central nervous system (CNS) in a mammalian subject, by administering to the subject a first agent that targets Wnt7a or Wnt7b that is present in the CNS and a second agent. For example, the subject may have a CNS tumor. CNS tumors include a neuronal tumor, a glial tumor, a meningeal tumor, a pituitary tumor, a pineal gland tumor, or a lymphatic tumor. The first agent is an inorganic molecule, peptide, peptide mimetic, antibody, liposome, small interfering RNA, antisense, aptamer, or external guide sequence, or a combination thereof. In one embodiment, the agent is a secreted Wnt 7a/7b signaling inhibitor. The second agent includes a radiotherapeutic or chemotherapeutic agent.

In yet another aspect, the invention provides a method of inducing angiogenesis in the central nervous system of a mammalian subject in need thereof, by administering a first agent that increases Wnt7a or Wnt7b activity in a CNS tissue. This method optionally further includes administering a second agent that stimulates angiogenesis, such as a growth factor or cytokine. In certain embodiments, the mammalian subject has suffered a stroke or a spinal cord injury. Alternatively, the mammalian subject is an infant exposed to perinatal hypoxia or ischemia. The first agent is an inorganic molecule, peptide, peptide mimetic, antibody, liposome, small interfering RNA, antisense, aptamer, or external guide sequence, or a combination thereof. The second agent is preferably an angiogenic growth factor, such as bone-derived angiogenic proteins (BDAPs), vascular endothelial cell growth factor (VEGF), basic fibroblast growth factor (bFGF), angiogenin, endothelial growth factor (EGF), platelet derived growth factor (PDGF), transforming growth factor-alpha (TGF-α), transforming growth factor-beta (TGF-β), or tumor necrosis factor-alpha (TNF-α).

Further features and advantages of the instant disclosed inventions will now be discussed in conjunction with the following Detailed Description, Drawings and Claims.

### BRIEF DESCRIPTION OF THE DRAWINGS

FIG. 1 depicts the nucleotide and amino acid sequences of the human Wnt7A protein, set forth as SEQ ID NOs: 1 and 2, respectively.

FIG. 2 depicts the nucleotide and amino acid sequences of the mouse Wnt7A protein, set forth as SEQ ID NOs: 3 and 4, respectively.

FIG. 3 depicts the nucleotide and amino acid sequences of the human Wnt7B protein, set forth as SEQ ID NOs: 5 and 6, respectively.

FIG. 4 depicts the nucleotide and amino acid sequences of the mouse Wnt7B protein, set forth as SEQ ID NOs: 7 and 8, respectively.

FIG. 5. Wnt ligands produced by the neuroepithelium are required for CNS vascularization. While Wnt7a+/+; Wnt7bc3/+ (A, F, K), Sox2Cre+/−; Wnt7a−/−; Wnt7bc3/+ (B, G, L), Sox2Cre+/−; Wnt7a+/−; Wnt7bc3/d3 (C, H, M) mutants appear phenotypically normal, Sox2Cre+/−; Wnt7a−/−; Wnt7bc3/d3 (D, I, N), and NestinCre+/−; Wnt7a−/−; Wnt7bc3/d3 (E, J, O) mutants exhibit a severe hemorrhaging restricted to the CNS as demonstrated by whole mount photos (A-E) and H&E stains (forebrain, F-J, and the forelimb level of the spinal cord, K-O). Furthermore, FLK1 (P-Z) and PDGFRb (A′-J′) immunohistochemistry for endothelial cells and pericytes, respectively, demonstrate that CNS vascularization is normal in Wnt7a+/+; Wnt7bc3/+ (P, U, A′, F′), Sox2Cre+/−; Wnt7a−/−; Wnt7bc3/+ (Q, V, B′, G′), Sox2Cre+/−; Wnt7a+/−; Wnt7bc3/d3 (R, X, C′, H′) mutants, but severely perturbed in Sox2Cre+/−; Wnt7a−/−; Wnt7bc3/d3 (S, Y, D′, I′), and to a lesser extent in NestinCre+/−; Wnt7a−/−; Wnt7bc3/d3 mutants (T, Z, E′, J′) at both the forebrain level (P-T, A′-E′) and the forelimb level of the spinal cord (U-Z, F′-I′).

FIG. 6. Canonical Wnt signaling in endothelial cells is required for CNS vascularization. While Flk1Cre^{+/-}; βCat^{c/wt} (A, E) embryos appear phenotypically normal, Flk1Cre^{+/-}; βCat^{c/n} (B, F) exhibit a severe hemorrhaging restricted to the CNS as demonstrated by whole mount photos (A, B) and H&E stains at the fore limb level of the spinal cord (E, F). Furthermore, FLK1 (I, J) and PDGFRb (M, N) immunohistochemistry for endothelial cells and pericytes, respectively, demonstrate that CNS vascularization is normal in Flk1Cre^{+/-}; βCat^{c/wt} (A, E), but severely perturbed in Flk1Cre^{+/-}; βCat^{c/n} (J, N).

FIG. 7. The blood-brain barrier marker GLUT1 is expressed in the PNVP and INVP in control embryos at E12.5 (A, B). In Sox2Cre+/−; Wnt7a−/−; Wnt7bc3/d3 double mutants, GLUT1 expression is severely downregulated, but some vascular segments express low levels of GLUT1 (D). In Flk1Cre+/−; βCatc/n, there is no expression in the endothelium. However, strong expression is detected in the neuroepithelium (E). In control embryos, GLUT1 expression is also detectable in the some vascular segments in the developing limb (C). However, the downregulation of GLUI1 in Flk1Cre+/−; βCatc/n mutants is specific for the CNS vasculature, the expression in the limbs is not perturbed (F). We targeted a construct allowing us to conditionally overexpress Wnt7a in a spatiotemporally controlled manner into the ROSA26 locus (G). Ectopic activation of Wnt7a outside the neural tube using the NestinCre allele resulted in a dramatic upregulation of GLUT1 in the vasculature outside the CNS(H, I).

FIG. 8. Neuroepithelial Wnt7a and Wnt7b expression is critical for CNS vascularization. Wnt7a^{+/+}; Wnt7b^{c3/+} (A-D), Sox2Cre^{+/-}; Wnt7a^{-/-}; Wnt7b^{c3/+} (E-H), Sox2Cre^{+/-}; Wnt7a^{+/-}; Wnt7b^{c3/d3} (I-L) mutants appear phenotypically normal at E12.5. In contrast, Sox2Cre^{+/-}; Wnt7a^{-/-}; Wnt7b^{c3/d3} (M-P), and NestinCre^{+/-}; Wnt7a^{-/-}; Wnt7b^{c3/d3} (Q-T) mutants exhibit a severe hemorrhaging that is restricted to the CNS as demonstrated by whole mount photos (A, E, I, M, Q) and a disruption of neural organization as demonstrated by H&E stains (B, F, J, N, R). Furthermore, FLK1 (C, G, K, O, S) and PDGFβ (D, H, L, P, T) immunohistochemistry for endothelial cells and pericytes, respectively, demonstrate that CNS vascularization is normal in Wnt7a^{+/+}; Wnt7b^{c3/+} (C-D), Sox2Cre^{+/-}; Wnt7a^{-/-}; Wnt7b^{c3/+} (G-H), Sox2Cre^{+/-}; Wnt7a^{+/-}; Wnt7b^{c3/d3} (K-L) mutants, but severely perturbed in Sox2Cre^{+/-}; Wnt7a^{-/-}; Wnt7b^{c3/d3} (O-P), and to a lesser extent in NestinCre^{+/-}; Wnt7a^{-/-}; Wnt7b^{c3/d3} mutants (S-T). In Sox2Cre^{+/-}; Wnt7a^{+/-}; Wnt7b^{c3/d3}, the ventral neural tube at spinal cord levels, with the exception of the area around the floor plate, is devoid of endothelial cells and pericytes (brackets in O, P). In the dorsal neural tube, endothelial cells and pericytes often form clusters or exhibit very large lumens (e.g., arrowheads in O). tel—telencephalon, di—diencephalon, mes—mesencephalon, rh—rhombencephalon, sc—Spinal cord, d—dorsal, v—ventral, NT—neural tube.

FIG. 9. Canonical Wnt signaling in endothelial cells is critical for CNS vascularization. BAT-gal mice report active canonical Wnt signaling (14). BAT-gal and Fli1-double positive endothelial cells were observed in the PNVP and INVP of the E10.5 mouse spinal cord (A, arrowheads in C, E, G). In addition, LEFT is also co-expressed in endothelial cells in the PNVP and INVP of the E10.5 mouse spinal cord (B, arrowheads in D, F, H). Flk1Cre-mediated removal of βCat results in the formation of a vascular system that can support the development of a mouse embryo until about E12, but as in Wnt7a/b double mutants a severe CNS-specific hemorrhaging phenotype (I, M) and a disruption of neural organization (J, N) is observed. FLK1 (K, O) and PDGFRβ (L, P) immunohistochemistry for endothelial cells and pericytes, respectively, demonstrate that a PNVP has formed in Flk1Cre^{+/-}; βCat^{c/n} embryos (arrowheads in 0, P), but that loss of vascular invasion is almost complete (O, P). Consistent with a direct model, removal of βCat from the neuroepithelium using the NestinCre allele results in no hemorrhaging at E12.5 (U-Y) and the PNVP and INVP are indistinguishable from control embryos (Q-T).

FIG. 10. Canonical Wnt signaling is necessary for proper differentiation of the CNS vasculature. The BBB marker GLUT-1 is present in the PNVP and INVP in control embryos at E12-12.5 (A-D, I-L, arrowhead in D points to a GLUT-1 positive vessel). In Sox2Cre^{+/-}; Wnt7a^{-/-}; Wnt7b^{c3/d3} double mutants, GLUT-1 is severely downregulated (E-H), but some vascular segments express low levels of GLUT-1 (arrowhead in E). Weak GLUT-1 remains in the ventral neuroepithelium, where vascular invasion fails (bracket in E, arrowhead in F). Boxes in A and E show the approximate region of B-D and F-H in separate sections, respectively. In Flk1Cre^{+/-}; βCat^{c/n} embryos, at E12 there is a dramatic reduction of GLUT-1 in the PNVP as compared to in control embryos (I-L, M-P, filled arrowhead in P). However, strong GLUT-1 is detected in the neuroepithelium (M-P, open arrowhead in P). Boxes in I and M show the approximate region of J-L and N-P in separate sections, respectively.

FIG. 11. Ectopic Wnt7a expression is sufficient to induce GLUT-1 in endothelial cells outside the CNS. NestinCre-mediated ectopic activation of a bicistronic message encoding for Wnt7a and LacZ in scattered cells outside the neural tube (A, E) resulted in a dramatic upregulation of GLUT-1 in the vascular endothelium outside the CNS (F-H) as compared to in control embryos (B-D).

FIG. 12. Wnt7a and Wnt7b have partially overlapping expression in the developing mouse neural tube. At E10.5, Wnt7a and Wnt7b expression is detected in a complementary pattern in the forebrain (A, B). However, at the forelimb level of the spinal cord, expression of Wnt7a and Wnt7b overlap (C, D). At this level, both genes are excluded from the dorsal fourth of the neural tube (brackets in C, D), which encompass the Wnt1, Wnt3a, and Wnt4 expression domains, and the floor plate (arrowheads in C, D), where Wnt4 is also expressed. Tel—Telencephalon, Di—Diencephalon, nt—neural tube.

FIG. 13. Neuroepithelial Wnt7a and Wnt7b expression is critical for CNS vascularization. Wnt7a^{+/+}; Wnt7b^{c3/+} (A-C), Sox2Cre^{+/-}; Wnt7a^{-/-}; Wnt7b^{c3/+} (D-F), Sox2Cre^{+/-}; Wnt7a^{+/-}; Wnt7b^{c3/d3} (G-I) mutants appear phenotypically normal. In contrast, Sox2Cre^{+/-}; Wnt7a^{-/-}; Wnt7b^{c3/d3} (J-L), and NestinCre^{+/-}; Wnt7a^{-/-}; Wnt7b^{c3/d3} (M-O) mutants exhibit a disruption of neural organization at E12.5 as demonstrated by H&E stains (e.g., filled arrowheads in J, M). Furthermore, FLK1 (B, E, H, K, N) and PDGFRβ (C, F, I, L, O) immunohistochemistry for endothelial cells and pericytes, respectively, demonstrate that CNS vascularization is normal in Wnt7a^{+/+}; Wnt7b^{c3/+} (B-C), Sox2Cre^{+/-}; Wnt7a^{-/-}; Wnt7b^{c3/+} (E-F), Sox2Cre^{+/-}; Wnt7a^{+/-}; Wnt7b^{c3/d3} (H-I) mutants, but severely perturbed in Sox2Cre^{+/-}; Wnt7a^{-/-}; Wnt7b^{c3/d3} (K-L), and to a lesser extent in NestinCre^{+/-}; Wnt7a^{-/-}; Wnt7b^{c3/d3} mutants (N-O). In Sox2Cre^{+/-}; Wnt7a^{-/-}; Wnt7b^{c3/d3} and NestinCre⁺^{/-}; Wnt7a^{-/-}; Wnt7b^{c3/d3} embryos, a PNVP has formed as endothelial cells and pericytes surround the neural tube (open arrowheads in K, L, N, O). In Sox2Cre^{+/-}; Wnt7a^{-/-}; Wnt7b^{c3/d3} embryos, no FLK1- or PDGFRβ-positive cells are detected in the pallium (brackets in K, L). Some invasion occurs in the MGE (and occasionally LGE) but the morphology of the vessels is perturbed (filled arrowheads in K, L). In NestinCre^{+/-}; Wnt7a^{-/-}; Wnt7b^{c3/d3} embryos, invasion occurs in all areas of the telencephalon, but the morphology of the vessels is perturbed (filled arrowheads in N, O). P—Pallium, LGE—lateral ganglionic eminence, MGE—medial ganglionic eminence, v—ventral, d—dorsal.

FIG. 14. The Nestin-Cre allele exhibits Cre recombinase activity in the neuroepithlium, but not in the CNS endothelium. Nestin-Cre^{+/-} driver males were crossed with gtROSA mice to provide a readout for Cre recombinase activity. X-Gal straining reveals lacZ expression in the neuroepithelium at both the level of the telencephalon and the forelimb (A, B). Double immunohistochemistry using antibodies directed against beta-gal (C, E, F, H) and the endothelial marker CD31 (D, E, G, H) reveal Cre recombinase activity in the neuroepithelium (open arrowheads in E, H), but not in the vascular endothelium (filled arrowheads in E, H). Boxes in A and B show the approximate region of C-E and F-H in adjacent sections, respectively.

FIG. 15. Temporal dynamics of NestinCre-mediated recombination in the CNS. Nestin-Cre^{+/-} driver males were crossed with gtROSA mice to provide a readout for Cre recombinase activity. X-Gal straining reveals lacZ expression in the neuroepithelium at E9.5 (left), E10.5 (middle), and E11.5 (right). Partial recombination is evident in the neural tube at E9.5 and E10.5.

FIG. 16. BAT-Gal reporter activity and LEF1 expression suggests a role for canonical Wnt signaling in CNS vascular development. The BAT-Gal mouse strain reports active canonical Wnt signaling in at least a subset of FLI1-positive endothelial cells in the PNVP and INVP of the E10.5 telencephalon (A, C, E, G). In addition, LEF1 is co-expressed in FLI1-positive endothelial cells in the PNVP and INVP of the E10.5 mouse telencephalon (B, D, F, H). MGE medial ganglionic eminence.

FIG. 17. The Flk1-Cre allele exhibits Cre recombinase activity in the CNS endothelium, but not in the neuroepithelium. Flk1-Cre^{+/-} driver males were crossed with gtROSA mice to provide a readout for Cre recombinase activity. X-Gal straining reveals lacZ expression in cells surrounding the neuroepithelium as well as in scattered cells within the neural tube at both the level of the telencephalon and the forelimb (A, B). Double immunohistochemistry using antibodies directed against beta-gal (C, E, F, H) and the endothelial marker CD31 (D, E, G, H) reveal Cre recombinase activity in the endothelium (arrowheads in E, H), but not in the neuroepithelium. Boxes in A and B show the approximate region of C-E and F-H in adjacent sections, respectively.

FIG. 18. Temporally regulated distribution of GLUT-1 in the neuroepithelium and CNS endothelium. (A-C) At E9.5, GLUT-1 is detected at high levels in the neuroepithlium at the forelimb level of the neural tube (bracket in A), but only a few FLK1-positive endothelial cells in the PNVP are GLUT-1 positive (arrowhead in C). (D-F) At E10.5, GLUT-1 is almost completely downregulated in the most ventral part of the neural tube at the forelimb level (open arrowhead in D); however, GLUT-1 persists, albeit at lower levels, in the dorsal neural tube (filled arrowhead in D). GLUT-1 is now abundant in endothelial cells in both the PNVP and the INVP (D-F). (G-I) At E11.5, GLUT-1 is restricted to the endothelium.

FIG. 19. Schematic of ROSA26-CAGGS-Wnt7a allele. The schematic illustrates the ROSA26-CAGGS-Wnt7a allele before and after Cre mediated recombination. (A) In the absence of Cre recombinase, expression from the CAGGS enhancer/promoter is terminated by three SV40 polyAs (tpA). (B) In the presence of Cre recombinase, the tpA is recombined out and the CAGGS enhancer/promoter drive expression of a bicistronic message encoding for Wnt7a and a nuclear β-galactosidase enzyme. A cassette containing a splice acceptor, the puromycin gene, and a polyA sequence precedes the CAGGS and was used to trap the ROSA26 locus.

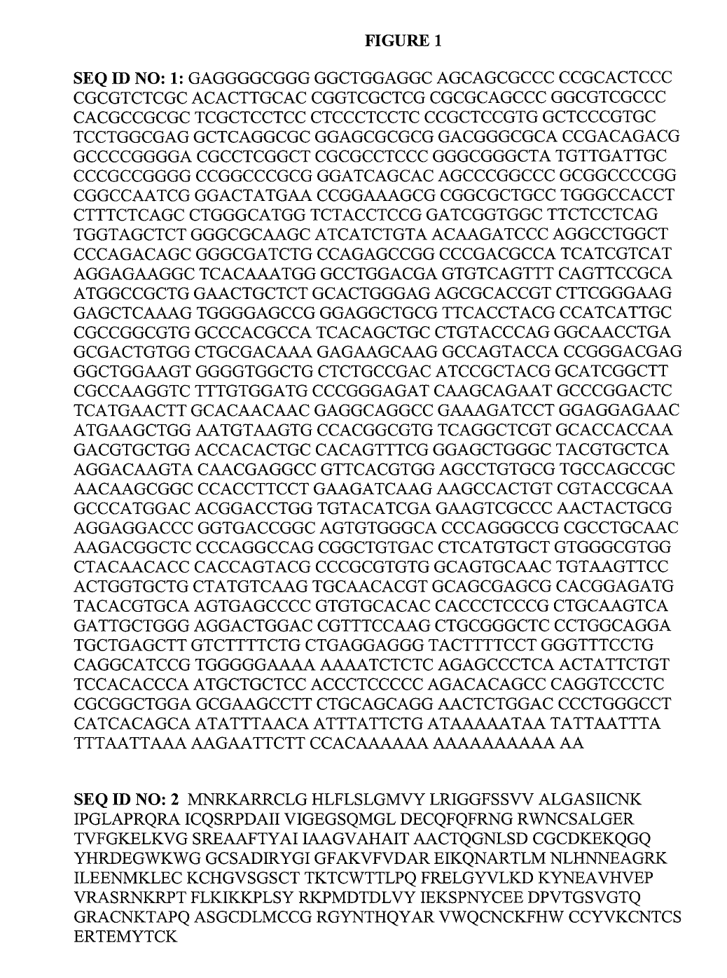

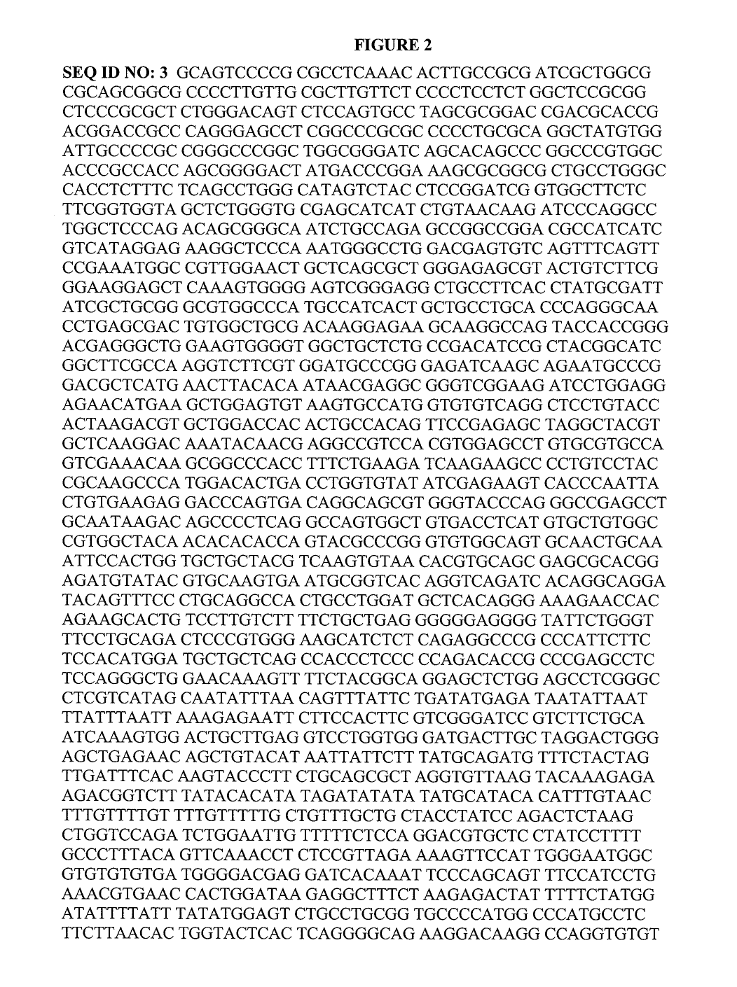

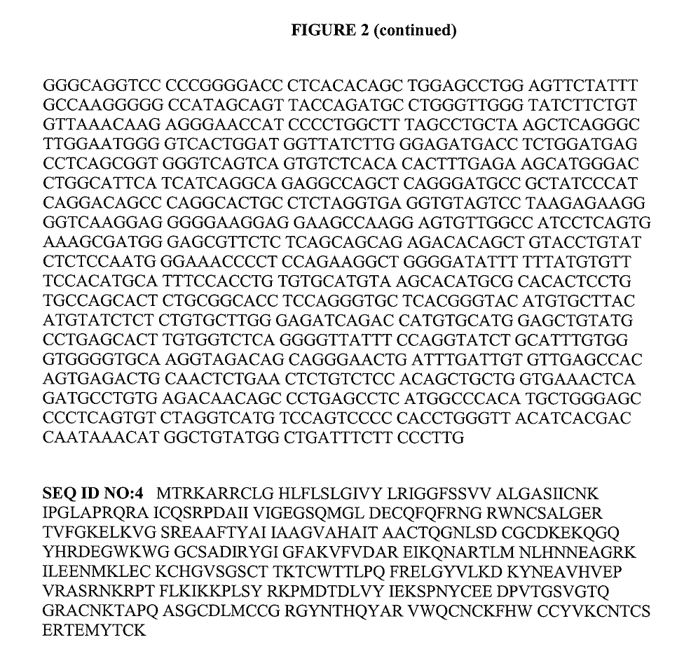

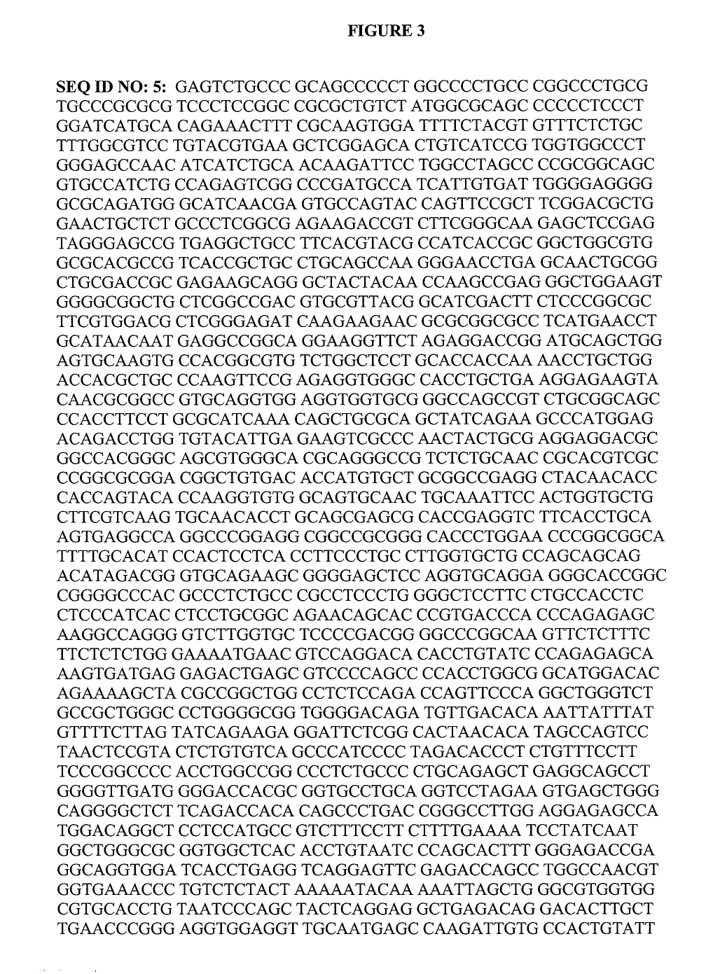

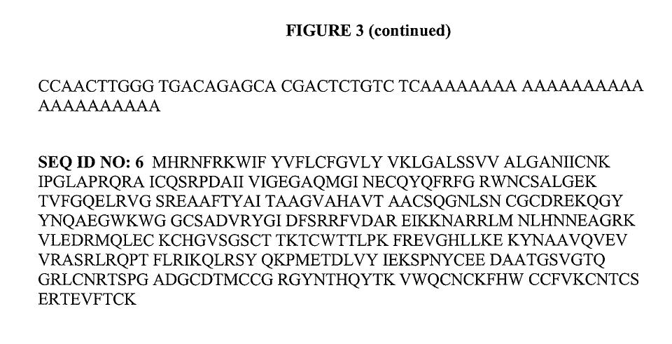

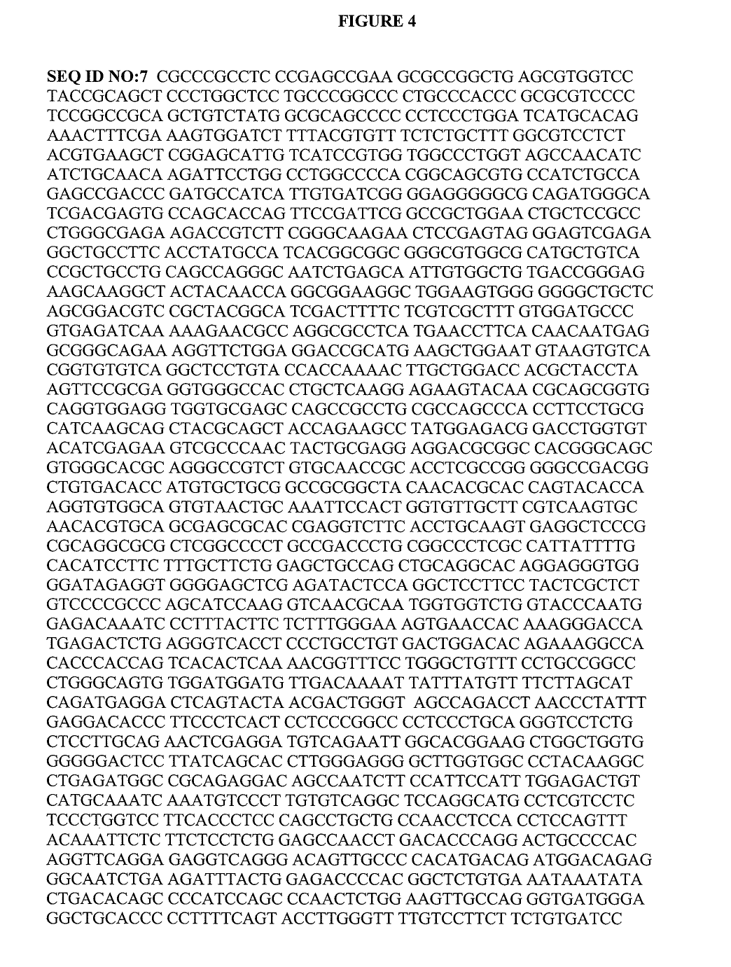

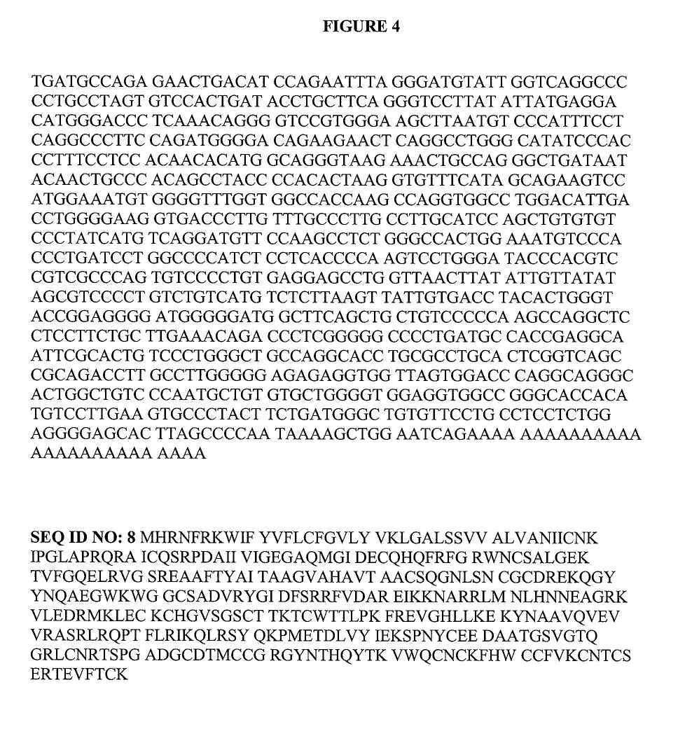

### DETAILED DESCRIPTION

### Definitions

As used herein, the following words and phrases have the meanings set forth below.

The singular forms “a”, “an”, and “the” include plural references unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “angiogenesis” refers to the formation of new blood vessels from a pre-existing vascular network.

The terms “cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, astrocytomas, ependymal tumors, glioblastoma multiforme (GBM), and primitive neuroectodermal tumors.

The “canonical Wnt pathway” or “canonical Wnt signaling” or “canonical Wnt signaling pathway” refers to a series of events that occur when Wnt ligands bind to cell-surface receptors of the Frizzled family, causing the receptors to activate Dishevelled family proteins and ultimately resulting in the nuclear translocation of beta-catenin where it associates with members of the TCF/LEF family and controls transcription of downstream target genes.

“Chemotherapy,” as used herein, refers to the use of chemicals, such as pharmaceuticals or drugs, in the treatment of a disease condition, such as cancer.

The term “component of the canonical Wnt signaling pathway” or “canonical Wnt signaling pathway component” refers to any of the proteins (receptors, ligands, etc.) that participate in canonical Wnt signaling as defined above. As used herein the term also includes components of other pathways associated with the canonical Wnt signaling pathway, such as components of the β-pathway. The terms “comprise” and “comprising” is used in the inclusive, open sense, meaning that additional elements may be included.

The term “disorders of the blood-brain barrier” refer to any disorder of the blood-brain barrier, including but not limited to, meningitis, multiple sclerosis, neuromyelitis optica, late-stage neurological trypanosomiasis, progressive multifocal leukoencephalopathy, De Vivo disease (also known as GLUT1 deficiency syndrome) and Alzheimer's disease.

The term “mammal” is known in the art, and exemplary mammals include humans, primates, bovines, porcines, canines, felines, and rodents (e.g., mice and rats).

The terms “modulate” and “modulation” refer to up regulation (i.e., increase, activation or stimulation), down regulation (i.e., decrease, inhibition or suppression) of a response, or the two in combination or apart. For example, an agent or a test compound can be considered to modulate a Wnt7a or Wnt7b activity if the presence of such agent or test compound in the assay of the invention results in either a decrease or increase in the assayed Wnt7a or Wnt7b activity.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The terms “parenteral administration” and “administered parenterally” refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

A “patient,” “subject” or “host” to be treated by the subject method may mean either a human or non-human animal.

The term “percent identity” refers to sequence identity between two amino acid or nucleotide sequences. Identity can each be determined by comparing a position in each sequence which may be aligned for purposes of comparison. When an equivalent position in the compared sequence is occupied by the same base or amino acid, then the molecules are identical at that position; when the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain applications a carrier is a polymer, an ultrasound-sensitive bio-polymer, a nano-particle cell, a micro-particle, a micelle, an ultrasound-sensitive stabilized pluronic micelle, a microbubble, a microsphere, or a microparticle made of insoluble or biodegradable natural or synthetic polymers. For other applications, the carrier is a biological material such as a cell, a cell ghost, a lipoprotein, or a liposome.

The term “prophylactic” or “therapeutic” treatment refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “small molecule” refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The terms “systemic administration,” “administered systemically,” “peripheral administration” and “administered peripherally” refer to the administration of a subject composition, therapeutic or other material other than directly into the central nervous system, such that it enters the patient's system and, thus, is subject to metabolism and other like processes.

The term “therapeutic agent” refers to any chemical moiety that is a biologically, physiologically, or pharmacologically active substance that acts locally or systemically in a subject. The term thus means any substance intended for use in the diagnosis, cure, mitigation, treatment or prevention of disease or in the enhancement of desirable physical or mental development and/or conditions in an animal or human.

The term “therapeutic effect” refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art.

The term “treating” refers to curing as well as ameliorating at least one symptom of any condition or disease or preventing a condition or disease from worsening.

“Tumor,” as used herein, refers to all neoplastic cell growth and proliferation, whether malignant or benign, and all pre-cancerous and cancerous cells and tissues.

The term “variant,” when used in the context of a polynucleotide sequence, may encompass a polynucleotide sequence related to that of a particular gene or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variation is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

A “variant” of a polypeptide refers to a polypeptide having the amino acid sequence of the polypeptide, but in which one or more amino acid residues have been altered. The variant may have “conservative” changes, wherein a substituted amino acid has similar structural or chemical properties (e.g., replacement of leucine with isoleucine). A variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “vasculogenesis” refers to the de novo formation of blood vessels from mesoderm.

The term “vascular disorders of the CNS” refer to any disorder associated with blood vessels that supply the CNS, including but not limited to, cerebral infarction, brain ischemia, brain hypoxia, intracranial embolism and thrombosis, intracranial arteriovenous malformations, etc. Blood vessels, include arteries and arterioles, veins and venules, and capillaries.

A “Wnt ligand” or “Wnt protein” is any member of a family of highly conserved secreted signaling molecules that regulate cell-to-cell interactions during embryogenesis. A list of Wnt ligands for various species is available at www.stanford.edu/rnusse/wntwindow.html. For example, the Wnt ligands (and the GenBank accession number for their transcript) in the mouse are Wnt1 (int-1, NM_{—}021279), Wnt2 (irp, NM_{—}023653), Wnt2b/13 (NM_{—}009520), Wnt3 (NM_{—}009521), Wnt3a (NM_{—}009522), Wnt4 (NM_{—}009523), Wnt5a (NM_{—}009524), Wnt5b (NM_{—}009525), Wnt6 (NM_{—}009526), Wnt7a (NM_{—}009527), Wnt7b (NM_{—}009528), Wnt8a (NM_{—}009290), Wnt8b (NM_{—}011720), Wnt9a (Wnt14, NM 139298), Wnt9b (Wnt15, NM_{—}011719), Wnt10a (NM_{—}009518), Wnt10b (NM_{—}011718), Wnt11 (NM_{—}009519), and Wnt16 (NM_{—}053116). The Wnt ligands (and the GenBank accession number for their transcript) in the human are Wnt1 (NM_{—}005430), Wnt2 (NM_{—}003391), Wnt2b/13 (NM_{—}024494 and NM_{—}004185), Wnt3 (NM_{—}030753), Wnt3a (NM_{—}033131), Wnt4 (NM_{—}030761), Wnt5a (NM_{—}003392), Wnt5b (NM_{—}032642), Wnt6 (NM_{—}006522), Wnt7a (NM_{—}004625), Wnt7b (NM_{—}058238), Wnt8a (NM_{—}058244), Wnt8b (NM_{—}003393), Wnt9a (Wnt14, NM_{—}003395), Wnt9b (Wnt15, NM_{—}003396), Wnt10a (NM_{—}025216), Wnt10b (NM_{—}003394), Wnt11 (NM_{—}004626) and Wnt16 (NM_{—}057168). Preferred Wnt ligands include Wnt ligands expressed in the CNS, including Wnt 1, Wnt 3, Wnt3a, Wnt4, and Wnt 5a, in addition to Wnt7a and Wnt7b.

“Wnt7a” refers to wingless-related MMTV integration site 7A. The nucleotide and amino acid sequences of the human protein are set forth as SEQ ID NOs: 1 and 2, respectively, and correspond to GenBank accession numbers NM_{—}004625 and NP_{—}004616, respectively. The nucleotide and the amino acid sequences of the mouse protein are set forth as SEQ ID NOs: 3 and 4, respectively and correspond to GenBank accession numbers NM_{—}009527 and NP_{—}033553, respectively.

“Wnt7b” refers to wingless-related MMTV integration site 7B. The nucleotide and amino acid sequences of the human protein are set forth as SEQ ID NOs: 5 and 6, respectively, and correspond to GenBank accession numbers NM_{—}058238 and NP_{—}478679, respectively. The nucleotide and the amino acid sequences of the mouse protein are set forth as SEQ ID NOs: 7 and 8, respectively and correspond to GenBank accession numbers NM_{—}009528 and NP_{—}033554, respectively.

### General

The molecular mechanisms controlling the coordinated development of an organ and its vasculature are poorly defined. Also, the type of endothelium formed depends on the nature of the organ. In the mouse, the central nervous system (CNS) vasculature forms by angiogenesis from the surrounding perineural vascular plexus (PNVP) and becomes highly specialized forming a tight barrier between the blood and the CNS, the blood-brain barrier (BBB). Using the developing CNS as a model system, we unexpectedly found that Wnt ligands produced by the developing neuroepithelium, particularly Wnt 7a and Wnt7b, act directly on the endothelium of the PNVP via the canonical pathway to both control CNS vascularization and establish properties of the BBB.

This finding suggests that Wnt signaling may be perturbed in diseases that affect the embryonic as well as adult vasculature and raises the possibility of treating such diseases by controlling the availability of Wnt ligand and thus the ability of the vascular endothelium to respond to such ligands. In addition, it would be possible to target the canonical pathway directly in the vascular endothelium by modulating the activity of components of the canonical Wnt signaling pathway, such as beta-catenin or members of the LEF/TCF family. As we have shown that Wnt ligands act directly on the endothelium to allow for angiogenesis to occur in the developing CNS, it is thus for example conceivable that such strategies could prevent angiogenesis associated with brain tumors and thus restrict the blood flow and therefore preventing the tumor from growing.

### Screening Assays to Identify Modulators of Wnt Ligands and Canonical Wnt Signaling Pathway Components

Based on the findings described in the exemplification, modulators of Wnt ligands and canonical Wnt signaling pathway components may be identified that may be used as therapeutics for vascular disorders of the CNS or disorders of the BBB. Such modulators may change the level of activity of the canonical Wnt signaling pathway in a responding endothelial cell, e.g., by modulating the activity of the Wnt ligands (by stimulating or preventing the production of Wnt ligands by neuroepithelial cells or by preventing or stimulating the binding to receptors on the target cells), or by targeting components of the canonical pathway inside the responding endothelial cell.

In general, agents or compounds capable of modulating Wnt ligands, in particular Wnt7a and Wnt7b, and the response to these ligands in CNS endothelial cells, e.g., via components in the canonical Wnt signaling pathway, can be identified using the instant disclosed assays to screen large libraries of both natural product or synthetic (or semi-synthetic) extracts or chemical libraries. Those skilled in the field of drug discovery and development will understand that the precise source of agents (e.g., test extracts or compounds) is not critical to the screening procedures of the invention. Accordingly, virtually any number of chemical extracts or compounds can be screened using the methods described herein. Examples of such agents, extracts, or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmnaMar, U.S.A. (Cambridge, Mass.). In addition, natural and synthetically produced libraries are produced, if desired, according to methods known in the art, for example, by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-viral activity should be employed whenever possible.

When a crude extract is found to modulate Wnt ligand activity, in particular Wnt7a and Wnt7b activity, or the activity of a component of the canonical Wnt signaling pathway, further fractionation of the positive lead extract is necessary to isolate chemical constituents responsible for the observed effect. Thus, the goal of the extraction, fractionation, and purification process is the careful characterization and identification of a chemical entity within the crude extract that modulates Wnt ligand or canonical Wnt signaling pathway component activity. Methods of fractionation and purification of such heterogeneous extracts are known in the art. If desired, compounds shown to be useful agents for modulating Wnt ligand or canonical Wnt signaling pathway component activity are chemically modified according to methods known in the art.

Potential modulators of Wnt ligand activity, in particular Wnt7a and Wnt7b activity, or canonical Wnt signaling pathway component activity, may include small molecules, small organic molecules, peptides, peptide mimetics, polypeptides, and antibodies that bind to a nucleic acid sequence or polypeptide of the invention and thereby stimulate or inhibit its activity. A modulator of a Wnt ligand, in particular a Wnt7a or Wnt7b ligand, or a canonical Wnt signaling pathway component, is any agent that changes or modulates the level or activity of the Wnt ligand or component, e.g. enhances, stimulates, activates, or inhibits, decreases, deactivates the level or activity of Wnt ligand or component, as the case may be.

In certain embodiments, a method for identifying an agent that modulates Wnt ligand activity or canonical Wnt signaling pathway component activity, comprises first contacting a Wnt ligand or component, or a portion thereof, with a test agent, and then determining the level of activity from the Wnt ligand, component or portion thereof in the presence of the test agent relative to the absence of the test agent. A change in activity indicates that the test agent is an agent that modulates the activity of Wnt ligand or component. Agents that stimulate the activity of a Wnt ligand would result in a higher level of activity of Wnt ligand or component in the presence of the test agent relative to the absence of the test agent. Likewise, agents that inhibit the activity of Wnt ligand or component would result in a lower level of activity of Wnt ligand or component in the presence of the test agent relative to the absence of the test agent.

The level of Wnt ligand activity or canonical Wnt signaling pathway component activity may be determined by measuring any assay for Wnt ligand activity or canonical Wnt signaling pathway component activity known to one of skill in the art.

For example, compounds that are targeted against a particular Wnt ligand or component may be assessed for the ability to modulate the Wnt signal transduction pathway, e.g., by assessing stabilization of proteins in the .beta.-catenin family as is described in Bahnot et al. for the armadillo (Arm) protein. Another way in which Wnt activity can be assessed by measuring activation of the T cell factor lymphoid enhancer factor (TcF-Lef) (Behrens et al., Nature 382:638-42, 1996). Active Wnt signaling results in other effects on cells such as phosphorylation of Dishevelled, or phosphorylation of the LRP tail. Wnt signaling may also be detected by reporter assays. For example, the TOP-flash assay is widely used and variants of TOP-Flash (SuperTop) are available from various sources (Upstate, the Moon lab).

In some situations it may be desirable to first screen candidate compounds for direct binding to the Wnt ligand or component or fragments thereof, in vitro, e.g., in a gel mobility shift assay and/or an immunoprecipitation assay, and then test Wnt ligand activity or canonical Wnt signaling pathway component activity in a second assay.

Assays to identify modulators of Wnt ligands or components may be cell based or in vitro assays. Cell based assays may utilize eukaryotic or mammalian cell lines known in the art, including but not limited to CHO cells, HeLa cells, Jurkat cells, Hep G2 cells, Cos cells, 3T3 cells, and PC12 cells. Wnt ligands may be fused to a reporter gene such as a gene expressing luciferase (Luc), green fluorescent protein (GFP), red fluorescent protein (RFP), yellow fluorescent protein (YFP), or cyan fluorescent protein (CFP). Enhanced forms of GFP and its derivatives may also be used as reporter genes.

Modulators of Wnt activity may be discovered using a cell that is known to respond to Wnt signaling (for example a target gene is activated as in the TOP/FLASH assay developed by Hans Clevers), wherein Wnt signal is measured in the presence and absence of a candidate modulator. The target or the modulator might be generic as in TOP/FLASH, which is an artificial target of canonical Wnt/bcatenin pathway, or more tailored to the cell type of interest such as Glut1 in an endothelial cell line.

In vitro based assays, e.g., may utilize techniques for immobilizing proteins and other molecules on matrices are also available for use in the subject assay. For instance, either Wnt ligand or an appropriate interacting molecule may be immobilized utilizing conjugation of biotin and streptavidin. For instance, biotinylated proteins can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with either Wnt ligand or an interacting molecule, but which do not interfere with the interaction between the polypeptide and the interacting molecule, can be derivatized to the wells of the plate, and may be trapped in the wells by antibody conjugation. As above, preparations of an interacting molecule and a test compound may be incubated in the polypeptide-presenting wells of the plate, and the amount of complex trapped in the well can be quantitated in the presence or absence of a test agent. Exemplary methods for detecting such complexes, in addition to those described for the GST-immobilized complexes, include immunodetection of complexes using antibodies reactive with the interacting molecule or enzyme-linked assays, which rely on detecting an enzymatic activity associated with the interacting molecule.

For example, an enzyme can be chemically conjugated or provided as a fusion protein with the interacting molecule. To illustrate, the interacting molecule can be chemically cross-linked or genetically fused with horseradish peroxidase, and the amount of polypeptide trapped in the complex can be assessed with a chromogenic substrate of the enzyme, for example, 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al. (1974) J. Biol. Chem. 249:7130).

Agents that function as activators of Wnt include compounds that bind to, and activate receptors of the Frizzled family on the cell surface, e.g. antibodies and fragments thereof, wnt mimetics and derivatives, and the like. Wnt inhibitors are agents that downregulate expression or activity of wnt. Agents of interest may interact directly with wnt, e.g. drugs, including small organic molecules, blocking antibodies, etc., or may interact with wnt associated proteins, e.g. Wnt co-receptors LRP5/6 and the transmembrane protein Kremen. Wnt inhibitors of interest interfere with the interaction between soluble, extracellular wnt proteins, and the frizzled receptors that are present on the surface of normal cells. Such agents include, e.g., soluble frizzled polypeptides comprising the wnt binding domains; soluble frizzled related polypeptides or proteins (sFRPs); wnt specific antibodies; frizzled specific antibodies; and other molecules capable of blocking extracellular wnt signaling. The Wnt7a and Wnt7b pathways are also antagonized by Dkks (Dickkopf-related proteins). See Kawano, Y. & R. Kypta (2003) J. Cell Science 116:2627. Exemplary Dkks are Dkk-1, Dkk-3, and Dkk-4.

An additional method of achieving Wnt modulation is the neutralization of a Wnt inhibitor, e.g., the chelation of Dkk by a soluble ectodomain of Kremen1/2 or LRP5/6).

### Methods For Treating Vascular Disorders of the CNS or Disorders of the Blood-Brain Barrier

In certain embodiments, methods for treating vascular disorders of the CNS or disorders of the blood-brain barrier may include increasing or decreasing the protein or activity level of Wnt ligands, in particular a Wnt7a or Wnt7b ligand, or a biologically active analog thereof in a cell of the subject. In certain embodiments, small molecules or agents that increase, e.g., enhance or stimulate, Wnt ligand expression can be used. In other embodiments, small molecules or agents that decrease, e.g., reduce or deactivate, Wnt ligand expression can be used. In other embodiments, recombinant or isolated purified versions of a Wnt ligand may be used to increase or decrease the level of Wnt ligands. For example, methods of producing recombinant Wnt7A ligands are known to those of skill in the art and are taught in U.S. Pat. Nos. 6,515,108 and 6,297,030.

In other embodiments, methods for treating vascular disorders of the CNS or disorders of the blood-brain barrier may comprise modulating the activity of components of the canonical Wnt signaling pathway in responding endothelial cells. In certain embodiments, small molecules or agents that increase, e.g., enhance or stimulate, component expression can be used. In other embodiments, small molecules or agents that decrease, e.g., reduce or deactivate, component expression can be used. In other embodiments, recombinant or isolated purified versions of a pathway component may be used to increase or decrease the level of Wnt ligands. Components that could be targeted in these methods, include but are not limited to, members of the Frizzled family, i.e., the receptors of the Wnt ligands (e.g., by preventing their expression in the target cells), beta-catenin (if beta-catenin is removed from endothelial cells of the CNS, then CNS vascularization does not occur and the blood brain barrier marker GLUT1 is missing, i.e. not expressed in the endothelium) and LEF/TCF family members (the expression of a dominant negative form of one of these family members could be used to repress the canonical pathway and thus angiogenesis in the CNS).

The efficacy of the agent can be assessed by generating dose response curves from data obtained using various concentrations of the test agent. Moreover, a control assay can also be performed to provide a baseline for comparison.

Methods for treating vascular disorders of the CNS or disorders of the blood-brain barrier in a subject comprise administering to the subject an effective amount of an activator of a Wnt ligand, wherein the activator of the Wnt ligand or component increases the level or activity of a Wnt ligand or component in cells of the subject, thereby treating the vascular disorder of the CNS or disorder of the blood-brain barrier in the subject.

An intact blood-brain barrier decreases or eliminates access of potential therapeutics to certain CNS diseases or disorders. Provided here are methods of treating a disease or disorder of the central nervous system in a mammalian subject, such as a human subject with a CNS disease or disorder, by administering to the subject two agents. The first agent targets Wnt7a or Wnt7b that is present in the CNS, thereby disrupting or otherwise affecting the blood-brain barrier. The second agent possesses one or more therapeutic properties useful in treating the CNS disease.

### Methods for Modulating Blood-Brain Barrier Permeability.

It has been demonstrated herein that Wnt7a and Wnt7b are critical determinants of the blood-brain barrier. Wnt7a and Wnt7b, and agents that target Wnt7a and/or Wnt7b that is/are present in the brain, are useful as modulators of the blood-brain barrier. An agent is administered such that it increases BBB permeability. Alternatively, an agent decreases BBB permeability. The increase or decrease of BBB permeability may be reversible, or irreversible. The agent is an inorganic molecule, peptide, peptide mimetic, antibody, liposome, small interfering RNA, antisense, aptamer, or external guide sequence.

In one embodiment, the agent is a secreted Wnt 7a/7b signaling inhibitor, such as a member of the Frizzled family (e.g., Fz1, Fz6). Roman-Roman et al., J Biol. Chem. (2004), 279:5725-5733; Golan et al., J Biol. Chem. (2004), 279:14879-14888.

Increased CNS vascular permeability are generally beneficial for treating CNS disorders, such as vasospasm of CNS blood vessels, Alzheimer's disease, Parkinson's disease, epilepsy, multiple sclerosis, schizophrenia, depression, stress, obesity, pain, anxiety, and any other CNS disorder that is directly or indirectly affected by BBB permeability changes. For example, increased CNS blood flow may improve the metabolic state of brain and other CNS tissue. Additionally, increased BBB permeability results in increased access to the CNS by drugs and other molecules generally excluded by the BBB. For example, radiotherapeutic or chemotherapeutic agents more effectively transverse the disrupted blood-brain barrier, resulting in increased efficacy of the radiotherapeutic or chemotherapeutic agent in the presence of a Wnt 7a/b modulator, as compared to in the absence of the Wnt 7a/b modulator.

### Methods for Treating CNS Tumors.

Additionally, brain and other CNS tumors require a blood supply to meet their increased metabolic demands, and therefore induce the development of tumor-associated vasculature. Pathologic angiogenesis, the process by which a malignant tumor develops and/or recruits new vessels, is the primary means by which cancer cells obtain a nutrient and oxygen source, and spread beyond the initial tumor situs. Proangiogenic factors are produced by tumor cells, stromal cells, or lymphocytes, which stimulate endothelial cell proliferation. Wnt7a/b modulation is useful to control tumor-associated angiogenesis and blood vessel remodeling, such as in a subject having a CNS tumor.

In one embodiment, agents that block Wnt7a and/or Wnt7b activity are introduced either proximal to the tumor, or systemically, to inhibit tumor vascularization and therefore, reduce or prevent the rate of tumor growth, and shrink tumor mass by “starving” the tumor. Wnt7a/b inhibitory agents may be provided with a second agent, such as a second anti-angiogenic compound (e.g., a VEGF blocker such as bevacizumab, angiostatin, endostatin, TNP-470, PI-88, etc.) that is known in the art. Alternatively, the second agent is a radiotherapeutic or chemotherapeutic agent.

It has been recognized that tumor-associated blood vessels typically lack intact cell-cell and cell-matrix interactions found in physiological angiogenesis and normal blood vessels. In a second embodiment of the invention, Wnt7a/b activators are provided to a tumor-associated blood vessel to induce endothelial cell differentiation, thereby reversing the pathophysiological state of the tumor blood vessels.

CNS tumors include a neuronal tumor, a glial tumor, a menigneal tumor, a pituitary tumor, a pineal gland tumor, and a lymphatic tumor. CNS tumors also include CNS metastases of systemic cancers (e.g., breast cancer, small cell lung cancer, lymphoma, and germ cell tumors).

Tumor tissue and/or cells may also be screened for Wnt independent growth. In such a method, tumor cells of interest are contacted with an agent that functions as a Wnt inhibitor, and the proliferation of the cells, e.g. by uptake of tritiated thymidine, etc., is monitored. In certain embodiments of the invention, a candidate subject is screened for the presence of a cancer that is associated with a mutation in the Wnt signaling pathway, so that patients having such a mutation are treated with the methods of the invention. Candidate cancers include, without limitation, those listed herein.

In addition to Wnt7a and Wnt7b, other CNS-expressed Wnts are targets for alteration in gene expression and/or activity. By way of non-limiting example, the compositions and methods of the invention include targeting Wnt 1, Wnt 3, Wnt3a, Wnt4, Wnt5a, and Wnt5b. In certain embodiments, the invention provides a first agent that modulates Wnt7a/7b, and a second agent the modulates a Wnt expressed in the CNS other than Wnt7a or Wnt7b (e.g., Wnt 1, Wnt 3, Wnt3a, Wnt4, Wnt5a, or Wnt5b).

The blood vessels formed by tumor-induced angiogenesis are typically pathophysiological in their cellular architecture, having abnormal cell-cell and cell-matrix interactions. Such abnormalities increase leakage of fluid into surrounding tissue, leading to cerebral edema, thereby increasing mortality and morbidity in brain tumor patients. A non-limiting use of the present invention is the providing of Wnt7a/b modulators that reduce cerebral edema in mammalian subjects suffering from a CNS tumor.

### Methods for Inducing Angiogenesis.

CNS tissue may become ischemic or hypoxic resulting from acute or chronic injury, or disease. For example, in a human who has suffered a stroke or a spinal cord injury, resulting in tissue injury from ischemia and/or hypoxia. Spinal cord injuries (SCI) include contusions (bruising of the spinal cord) and compression injuries (caused by pressure on the spinal cord). In another example, an infant exposed to perinatal hypoxia or ischemia suffers tissue damage.

The invention provides a method of inducing angiogenesis in the central nervous system of a mammalian subject in need thereof, by administering a first agent that increases Wnt7a or Wnt7b activity in a CNS tissue, therefore increasing angiogenesis in that tissue.

This method optionally further includes administering a second agent that stimulates angiogenesis, such as a growth factor (i.e., “an angiogenic growth factor”) or cytokine Preferred angiogenic growth factors include bone-derived angiogenic proteins (BDAPs), vascular endothelial cell growth factor (VEGF), basic fibroblast growth factor (bFGF), angiogenin, endothelial growth factor (EGF), platelet derived growth factor (PDGF), transforming growth factor-alpha (TGF-α), transforming growth factor-beta (TGF-β), and tumor necrosis factor-alpha (TNF-α). (Freedman, S. B., and Isner, J. M., Therapeutic angiogenesis for ischemic cardiovascular disease, J. Mol. Cell. Cardiol 33(3): 379-393 (2001)).

### Alteration of Wnt Gene Expression.

The level of protein can also be increased in a cell, e.g., by introducing into the cell a nucleic acid encoding the protein operably linked to a transcriptional regulatory sequence directing the expression of the protein in the cell.

Methods for expressing Wnt ligand or component nucleic acids in cells and appropriate transcriptional regulatory elements for doing so are well known in the art.

Alternatively, a protein can be introduced into a cell, usually in the presence of a vector facilitating the entry of the protein into the cells, e.g., liposomes. Proteins can also be linked to transcytosis peptides for that purpose. Yet in other methods, an agent that stimulates expression of the endogenous gene is contacted with a cell. Such agents can be identified as further described herein.

Any means for the introduction of polynucleotides into mammals, human or non-human, or cells thereof may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences). Formulation of DNA, e.g. with various lipid or liposome materials, may then be effected using known methods and materials and delivered to the recipient mammal.

DNA constructs may also be delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans.

The expression of a protein, e.g., a Wnt ligand or a biologically active variant thereof in cells of a subject to whom, e.g., a nucleic acid encoding the protein was administered, can be determined, e.g., by obtaining a sample of the cells of the patient and determining the level of the protein in the sample, relative to a control sample.

### Diagnostics to Monitor Vascular Disorders of the CNS or Disorders of the Blood-Brain Barrier

Methods for monitoring the severity of vascular disorders of the CNS or disorders of the blood-brain barrier may comprise measuring the expression of Wnt ligands, canonical Wnt signaling pathway components, or the level of canonical Wnt signaling that occurs in the endothelium. For example, measuring the activity of the canonical Wnt signaling pathway active in the vasculature of brain tumors may be useful in staging the cancer. As described herein, Wnt ligands, including at least Wnt7a and Wnt7b, act directly on the endothelium of the PNVP and INVP via the canonical pathway controlling CNS vascularization and the establishment of some BBB properties. Thus, expression levels of Wnt ligands or the ectopic activation/or downregulation of canonical Wnt signaling pathway activity in the responding cell population may correlate to the stages and severity of vascular disorders of the CNS or disorders of the blood-brain barrier. Activity can be monitored using the various approaches described herein.

Alternatively, the expression of polypeptides encoded by Wnt ligand genes may be monitored, e.g., using antibody-based methods such as immunoassays. Any suitable immunoassay can be used, including, without limitation, competitive and non-competitive assay systems using techniques such as western blots, radioimmunoassays, ELISA (enzyme-linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

### Compositions of Modulators of Wnt Ligands and Recombinant or Purified Wnt Ligands

Compounds identified through the screening assays described above as well as Wnt ligand and canonical Wnt signaling component nucleic acids and proteins can be administered to a subject according to methods known in the art. For example, nucleic acids encoding a Wnt ligand or canonical Wnt signaling component or an antisense molecule can be administered to a subject as described above, e.g., using a viral vector.

Pharmaceutical agents for use in accordance with the present methods may be formulated in conventional manner using one or more physiologically acceptable carriers or excipients. Thus, proteins and nucleic acids described herein as well as compounds or agents that increase the protein or expression level of nucleic acids described herein, and their physiologically acceptable salts and solvates may be formulated for administration by, for example, injection, inhalation or insufflation (either through the mouth or the nose) or oral, buccal, parenteral or rectal administration. In one embodiment, the agent is administered locally, e.g., at the site where the target cells are present, such as by the use of a patch.

Agents can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Agents that may oxidize and lose biological activity, especially in a liquid or semi-solid form, may be prepared in a nitrogen atmosphere or sealed in a type of capsule and/or foil package that excludes oxygen (e.g. Capsugel™).

For administration by inhalation, the agents may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the agent and a suitable powder base such as lactose or starch.

The agents may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The agents may also be formulated in rectal compositions such as suppositories or retention enemas, e.g., containing conventional suppository bases such as cocoa butter or other glycerides.

In addition to the formulations described previously, the agents may also be formulated as a depot preparation. Such long acting formulations may be administered by implantation (for example subcutaneously or intramuscularly) or by intramuscular injection. Thus, for example, the agents may be formulated with suitable polymeric or hydrophobic materials (for example as an emulsion in an acceptable oil) or ion exchange resins, or as sparingly soluble derivatives, for example, as a sparingly soluble salt. Controlled release formula also include patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions (including cosmetic preparations) may comprise from about 0.00001 to 100% such as from 0.001 to 10% or from 0.1% to 5% by weight of one or more agents described herein.

In one embodiment, an agent described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Animal-based models, such as those described in the Exemplification, may also be used to identify compounds capable of ameliorating vascular disorders of the CNS or disorders of the blood-brain barrier. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate vascular disorders of the CNS or disorders of the blood-brain barrier, at a sufficient concentration and for a time sufficient to elicit such an amelioration of vascular disorders of the CNS or disorders of the blood-brain barrier in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the infection or phenotype. Neonatal, juvenile, and adult animals can also be exposed.

In one embodiment, the present invention provides a method of identifying agents activating Wnt ligand expression or function. The method includes measuring a physiological response of the animal, for example, to the agent, and comparing the physiological response of such animal to a control animal, wherein the physiological response of the wild-type animal compared with an animal comprising a disruption or knock-out of the Wnt ligand gene indicates the specificity of the agent. A “physiological response” is any biological or physical parameter of an animal that can be measured. Molecular assays (e.g., gene transcription, protein production and degradation rates), physical parameters (e.g., exercise physiology tests, measurement of various parameters of respiration, measurement of heart rate or blood pressure, measurement of bleeding time), behavioral testing, and cellular assays (e.g., immunohistochemical assays of cell surface markers, or the ability of cells to aggregate or proliferate) can be used to assess a physiological response.

### Drug Delivery

Further provided are compositions and methods for delivering therapeutics across the BBB. We have discovered that the canonical Wnt signaling pathway controls the development and likely the integrity of the BBB. Thus, the compositions of modulators of Wnt ligands and recombinant or purified Wnt ligands described above may be used to weaken the BBB, e.g., by loosening the tight junctions between the endothelial cells of the brain's capillaries by affecting the vascularization of the BBB.

Accordingly, in certain embodiments, the compositions of modulators of Wnt ligands and recombinant or purified Wnt ligands described above may be administered to a subject in need thereof in order to make the BBB more permeable or able to be traversed by at least one therapeutic agent. In certain embodiments, one or more of the compositions may be administered to a subject prior to administering the at least one therapeutic agent. In other embodiments, one or more of the compositions may be administered to a subject simultaneously with at least one therapeutic agent. In still other embodiments, one or more of the compositions may be coupled to the therapeutic agent by any means known to one of skill in the art. The dose of the compositions may be determined by one of skill in the art as that which causes a temporary weakening of the BBB so as to allow the therapeutic to pass through.

### Additional Embodiments

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims. Although this disclosure has been described in terms of certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXEMPLIFICATION

The present invention is further illustrated by the following examples, which should not be construed as limiting in any way.

### Example 1

### Generation and Characterization of Wnt7a; Wnt7b (Wnt7a/b) Double Mutants

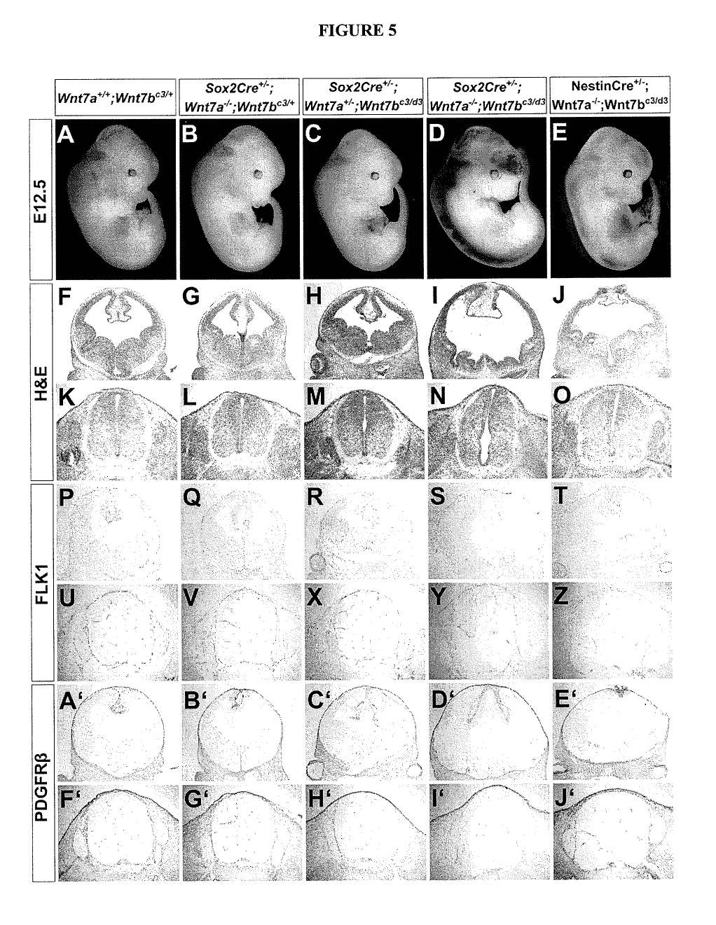

To examine a potential role for Wnt signaling in CNS vascularization, we generated Wnt7a; Wnt7b (Wnt7a/b) double mutants by using a conditional allele allowing us to circumvent the placental defects previously reported (Parr, 2001 #6). Mice double heterozygous for Wnt7a and Wnt7b null alleles were bred to Sox2-Cre (S2C) mice to generate driver males allowing us to selectively remove Wnt7b on a Wnt7a mutant background in the epiblast (Parr, 1998 #9; Parr, 2001 #6; Hayashi, 2002 #7). While Wnt7a and Wnt7b single mutants appear phenotypically normal at E12.5, S2C; Wnt7a/b double mutants exhibit a hemorrhaging phenotype that is restricted to the CNS (FIG. 5, data not shown). In addition, removing one Wnt7a allele on a Wnt7b mutant background and in most instances one Wnt7b allele on a Wnt7b mutant background reveals no phenotype at E12.5 (FIG. 5). In the few Wnt7a−/−; Wnt7bd3/+ mutants that exhibit hemorrhaging at E12.5, the phenotype is dramatically milder than the S2C; Wnt7a/b double mutant phenotype (data not shown).

A detailed molecular analysis using antibodies directed against the general endothelial markers Flk1, PECAM, Endoglin, Tie2, VE-Cadherin, and the pericyte marker PDGFR-b, reveal that the presence of these cells types in the PNVP and the neuroepithelium at both the level of the telencephalon and the forelimb. At the forebrain level, no endothelial cells or pericytes are detected in the pallium or lateral ganglionic eminence. A few cells are detected in the medial ganglionic eminence, but these tend to form a cluster of cells rather than a patent vascular plexus. At the spinal cord level, endothelial cells and pericytes are present in the PNVP, but the ventral neural tube with exception of the area around the floor plate, is devoid of endothelial cells and pericytes. It is interesting to note that Wnt4 is expressed in the dorsal neural tube as well as in the floor plate, suggesting that other Wnt ligands may partially compensate for the loss of Wnt7a/b. The endothelial cells that invade the neuroepithelium occasionally form clusters of endothelial cells and pericytes. In addition, vessels with very large lumen have been observed. The vessels midway along the dorsal-ventral axis tend to appear more abnormal than vessels in the dorsal neural tube.

As it possible that endothelial cells express low levels of Wnt7a/b, we wanted to confirm that the Wnt7a/b double mutant phenotype is a result of Wnt signaling from the neuroepithelium. A Nestin-Cre (NC) allele that drives expression of Cre recombinase in the neuroepithelium but not in blood vessels has been reported (Graus-Porta, 2001 #12; Tronche, 1999 #8). Thus, we bred mice double heterozygous for Wnt7a and Wnt7b null alleles to NC mice to generate driver males allowing us to selectively remove Wnt7b on a Wnt7a mutant background in the neuroepithelium. As only little recombination has occurred by E9.5 in this model (Backman, 2005 #13), some Wnt7b expression would be expected thus potentially resulting in a less severe phenotype than in the S2C; Wnt7a/b model. Indeed, at E12.5, we observe CNS specific hemorrhaging in the NC; Wnt7a/b double mutants (FIG. 5). A molecular analysis at E12.5 of the telencephalon and the forelimb level of the spinal cord reveal the presence of endothelial cells in the PNVP and neuroepithlium expressing Flk1, CD31, CD34, Tie2 and PDGFRb as well as pericytes expressing PDGFRb. The number of endothelial cells and pericytes appears reduced and the morphology of the vessels making up the INVP is clearly abnormal.

Thus far, we have established that Wnt ligands produced by the neuroepithelium are required for CNS vascularization, but the Wnt ligands could act indirectly via the neuroepithlium or directly on the endothelium and activate either the canonical or non-canonical pathways. Interestingly, bat-gal mice report active canonical Wnt signaling in endothelial cells of the PNVP and INVP (Maretto, 2003 #14), and, in addition, LEFT is expressed in endothelial cells of the PNVP and INVP, suggesting that the Wnt ligands may act directly on the endothelium via the canonical pathway. To test this possibility, we removed beta-catenin, which is essential for canonical Wnt signaling specifically in endothelial cells using a Flk1-Cre (FC) driver line (Motoike, 2003 #10).

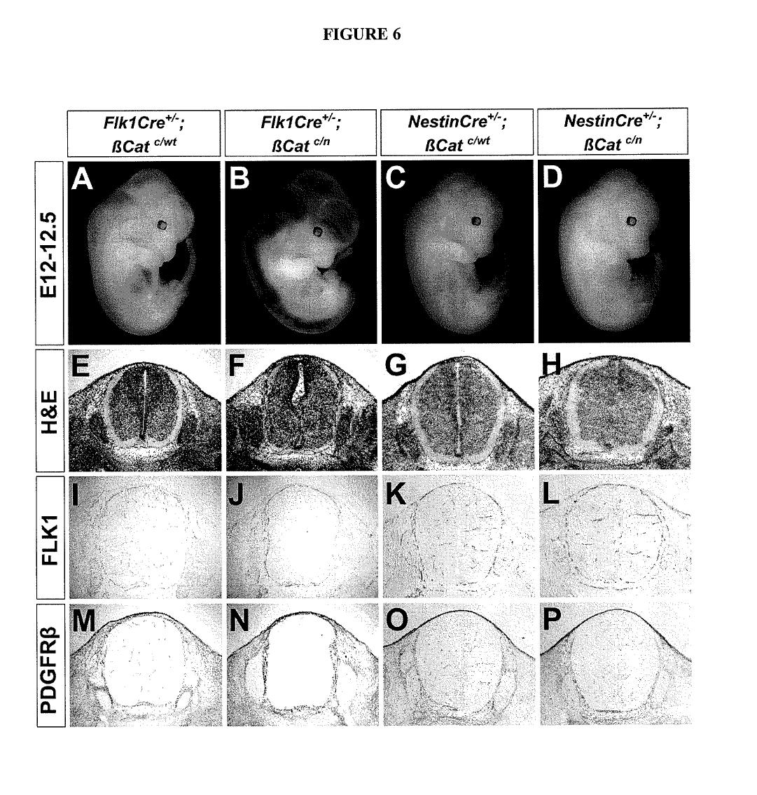

Interestingly, the removal of beta catenin specifically in the endothelial lineage results in a phenotype very similar to that of Wnt7a; Wnt7b double mutant embryos. While control embryos appear phenotypically normal at E12.5, endothelial specific beta catenin mutants exhibit a severe hemorrhaging phenotype apparently restricted to the CNS (FIG. 6). A detailed molecular analysis using antibodies that detect endothelial cells and pericytes show that while the perineural vascular plexus forms, the CNS is completely avascular (FIG. 6). This result together with the Wnt7a; Wnt7b double mutant results, suggest strongly that Wnt7a and Wnt7b produced by the neuroepithelium act directly on the endothelium of the PNVP to control CNS vascularization likely via the canonical Wnt signaling pathway.

We also removed beta-catenin from the neuroepithelium using the NC allele. Consistent with the direct model, we observed no hemorrhaging at E12.5 and the PNVP and INVP are indistinguishable from control embryos (FIG. 6).

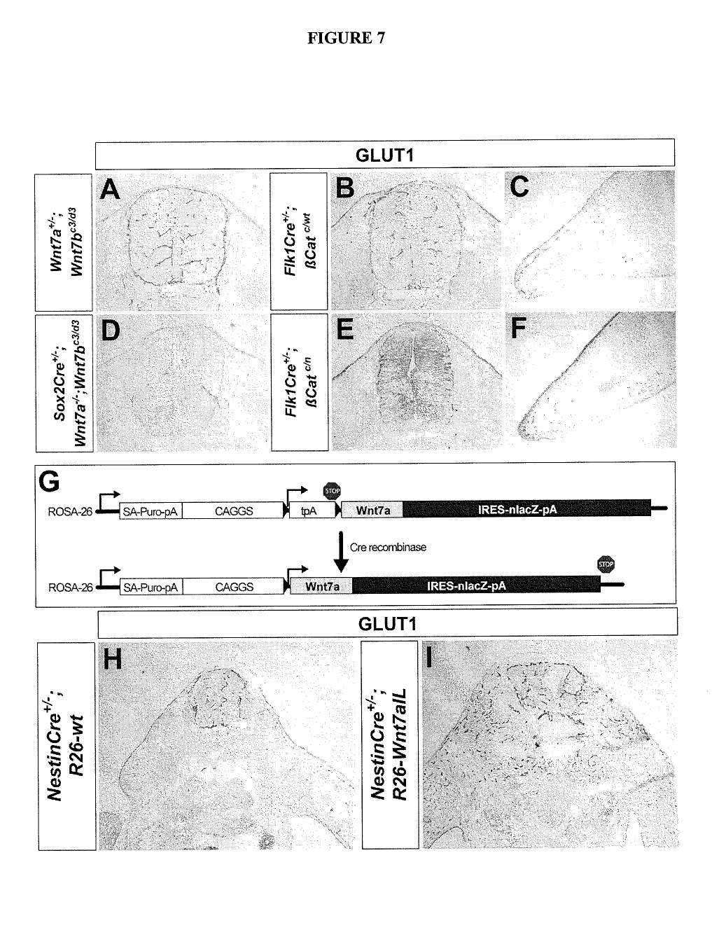

In order to address whether, in addition to the CNS angiogenesis defects we observed, the differentiation of the endothelial cells of the PNVP appear normal, we analyzed the expression of several general endothelial markers (such as Endoglin, Tie2) and all these appeared normal. In addition, we have analyzed the expression of the glucose transporter protein Glut1 which normally is expressed in the developing neuroepithlium prior to vascular invasion. After vascular invasion begins, the neuroepithlial expression of Glut1 is downregulated and Glut1 expression is upregulated in the PNVP and INVP endothelium. Expression of Glut1 can also be detected in the developing limbs, however, Glut1 expression is highly enriched in the CNS vasculature vs other endothelia and is considered as a good marker of the blood-brain barrier. Interestingly, in Wnt7a; Wnt7b double mutants, Glut1 expression is severely downreguated in the CNS vasculature (FIG. 7). Some low level expression is detected in some of the few vessels that develop in the dorsal part of the neural tube which is consistent with low level Wnt signaling still occurring in the dorsal neural tube. However, in Flk1Cre+/−; bcatc/n mutants, Glut1 expression is completely gone in the PNVP and expression is now detected in the neuroepithelium (FIG. 7). In addition, we targeted a construct allowing us to ectopically expresse Wnt7a outside of the neural tube using an appropriate Cre drvier line into the ROSA26 locus (FIG. 7). Ectopic expression of Wnt7a outside of the neural tube using the NestinCre allele demonstrate that expression of Wnt7a is sufficient to induce ectopic expression of Glut1 in at least some other endothelial cells (FIG. 7). Thus, it appears reasonable to conclude that Wnt7a and Wnt7b act via the canonical pathway to induce Glut1 expression in the blood-brain barrier.

In summary, our data argues strongly that Wnt ligands, including at least Wnt7a and Wnt7b, act directly on the endothelium of the PNVP and INVP via the canonical pathway controlling CNS vascularization and the establishment of the BBB marker Glut1. Interestingly, beta-catenin has previously been removed specifically in endothelial cells, but the authors did not report hemorrhaging restricted to the CNS. Rather than as a mediator of canonical Wnt signaling, Cattelino et al. suggested that beta-catenin functions in the endothelium to stabilize adherens junctions (Cattelino, 2003 #11). Our data argues strongly against the findings of Cattelino et al.

Several signaling pathways controlling vasculogenesis and angiogenesis have been defined, however, less is known about the molecular mechanisms controlling the coordinated development of an organ and its vasculature. We suggest that CNS development and vascularization is coordinated by neuroepithelial-derived Wnt ligands controlling directly aspects of PNVP and INVP differentiation via the canonical pathway. We have shown that Wnt activity controls the expression of the BBB marker Glut1, thus at least partially controlling the type of endothelium formed in the CNS.
1. C. L. Farrell, W. Risau, Microsc Res Tech 27, 495 (1994).
2. K. A. Hogan, C. A. Ambler, D. L. Chapman, V. L. Bautch, Development 131, 1503 (2004).
3. B. Engelhardt, Cell Tissue Res 314, 119 (2003).
4. B. A. Parr, M. J. Shea, G. Vassileva, A. P. McMahon, Development 119, 247 (1993).
5. B. A. Parr, V. A. Cornish, M. I. Cybulsky, A. P. McMahon, Dev Biol 237, 324 (2001).
6. B. A. Parr, A. P. McMahon, Nature 395, 707 (1998).
7. S. Hayashi, P. Lewis, L. Pevny, A. P. McMahon, Mech Dev 119 Suppl 1, S97 (2002).
8. D. Graus-Porta et al., Neuron 31, 367 (2001).
9. F. Tronche et al., Nat Genet. 23, 99 (1999).
10. M. Backman et al., Dev Biol 279, 155 (2005).
11. S. Maretto et al., Proc Natl Acad Sci USA 100, 3299 (2003).
12. T. Motoike, D. W. Markham, J. Rossant, T. N. Sato, Genesis 35, 153 (2003).
13. A. Cattelino et al., J Cell Biol 162, 1111 (2003).
14. Gavin, et al., Genes Dev. 4, 2319-32 (1990).

### Example 2

### Vasculogenesis and Angiogenesis of the Central Nervous System is Directly Controlled by Wnt7a and Wnt7b Produced by Neuroepithelium

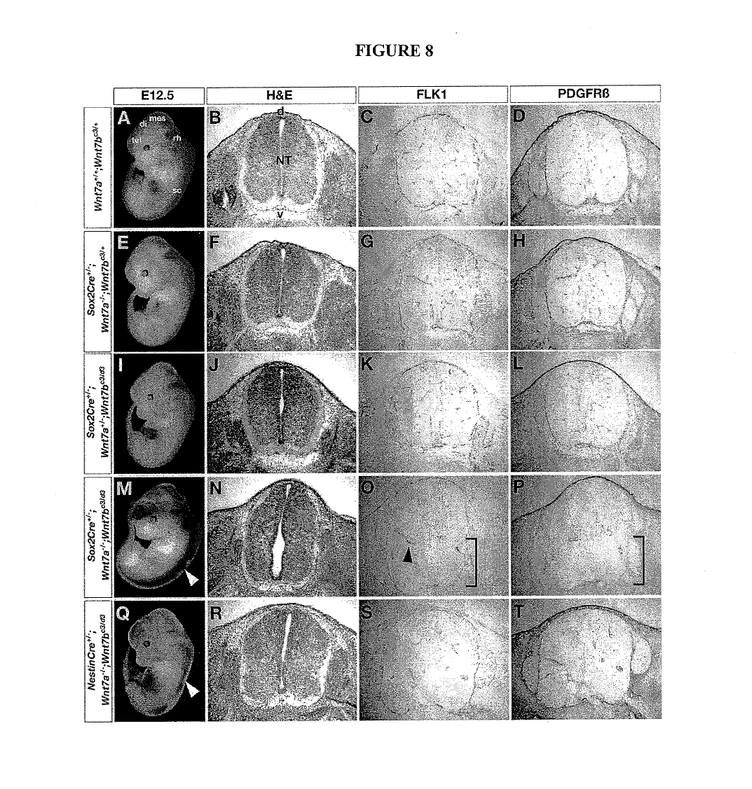

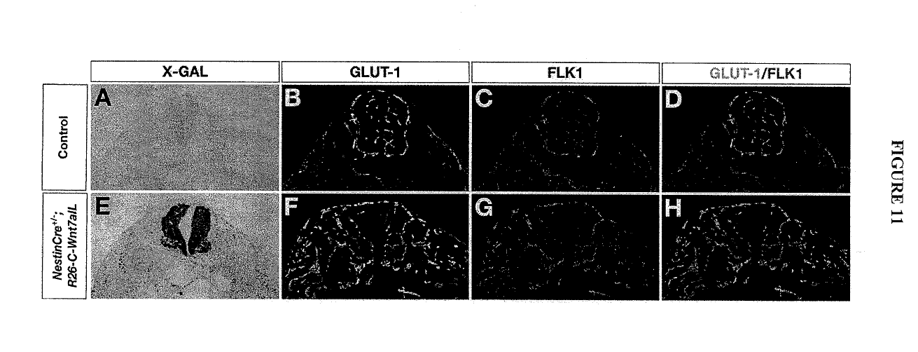

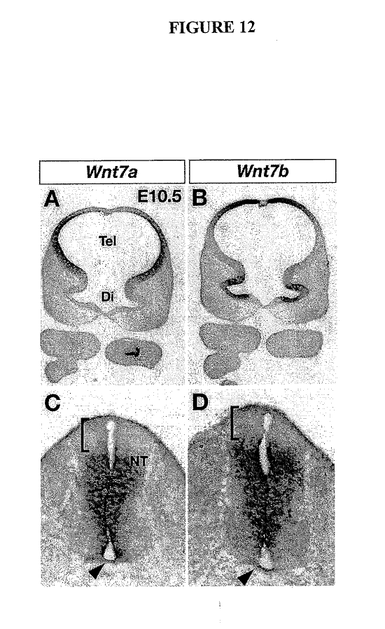

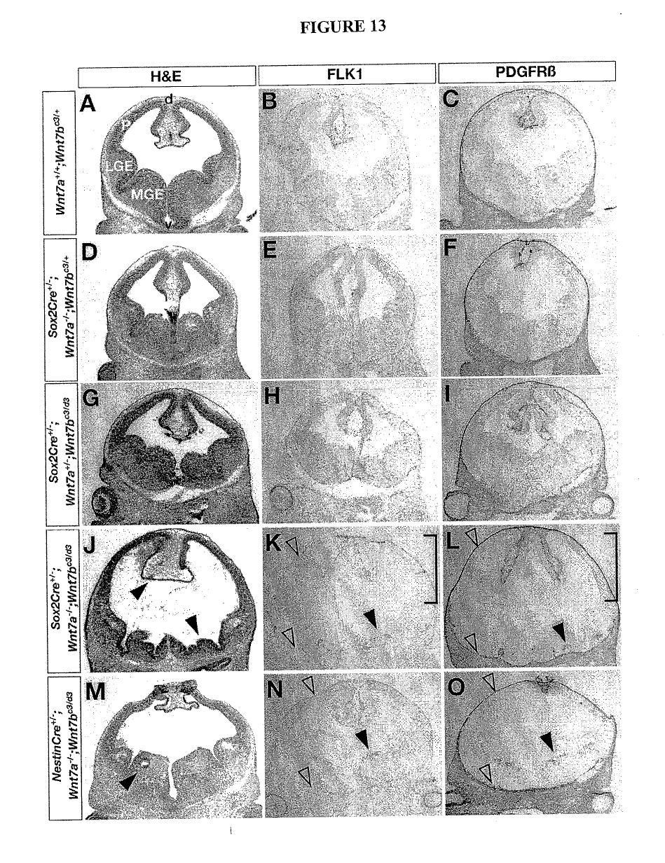

Expression of both Wnt7a and Wnt7b can be detected in the neural tube at E9.5 coincident with CNS vascular development. At E10.5, Wnt7a and Wnt7b are expressed in broad overlapping domains along the dorsal-ventral axis in the spinal cord; however, at the forebrain level they are expressed in a complementary pattern (FIG. 12). Null mutants for both Wnt7a and Wnt7b have been generated, neither one of which exhibit an early neural tube phenotype. As described, herein, a conditional null allele of Wnt7b has been generated that, in combination with a Sox2Cre transgenic allele, confines Wnt7b removal to the embryo enabling an earlier placental phenotype to be overcome. These mice were crossed with a Wnt7a null allele to generate Sox2Cre; Wnt7a/b double mutants. When examined at E12.5, embryos lacking embryonic Wnt7a and Wnt7b signaling exhibit a hemorrhaging phenotype that is restricted to the CNS (FIGS. 8A, E, I, M, data not shown), the first functional evidence that Wnt signaling may be involved in CNS vascular development. In most instances, when a single active Wnt7a or Wnt7b allele remained, no phenotype was observed. However, a small number of Wnt7a^{-/-}; Wnt7b^{d3/+} mutants exhibit a mild hemorrhaging phenotype at E12.5 (data not shown). Histological analysis reveals a disruption of neural organization at both the spinal cord level and forebrain; however, as these defects are not evident at E10.5, they are most likely secondary to the hemorrhaging phenotype (FIG. 8B, F, J, N, FIG. 13, and data not shown). No obvious differences were observed in neural patterning (regionally restricted Nkx2.2, Pax6 and Pax7) or VEGF localization between control embryos and Wnt7a/b double mutants at E10.5 (data not shown).

In order to visualize vascular cells, we performed a detailed molecular analysis using antibodies directed against the general endothelial markers FLK1, CD31, Endoglin, TIE2, VE-Cadherin, and the pericyte marker PDGFRβ focusing on the presumptive spinal cord forelimb level (FIG. 8C-D, G-H, K-L, O-P, and data not shown) and forebrain (FIG. 13) at E11.5 and E12.5. Consistent with normal VEGF signaling, a PNVP forms in Wnt7a/b double mutants, endothelial cells and pericytes are evident surrounding the neural tube (FIG. 8O, P). However, with the exception of the area around the floor plate, endothelial cells and pericytes are absent within the ventral neural tube at spinal cord levels (FIG. 8O, P). Vascular segments are evident in the dorsal part of the neural tube but often these form abnormally large clusters of endothelial cells and pericytes or exhibit very large lumens (e.g., arrowhead in FIG. 8O). Interestingly, vessels at the dorsal-ventral intersect appear more abnormal than vessels in the most dorsal part of the neural tube. The dorsal neural tube expresses Wnt1, 3, 3a at the dorsal midline, and Wnt4 more broadly (7). Thus, the most severe phenotypes are observed in regions where Wnt7a and Wnt7b are the predominant Wnt inputs. The milder phenotypes elsewhere in the CNS argue for a more general role for other Wnt factors in CNS vascularization.

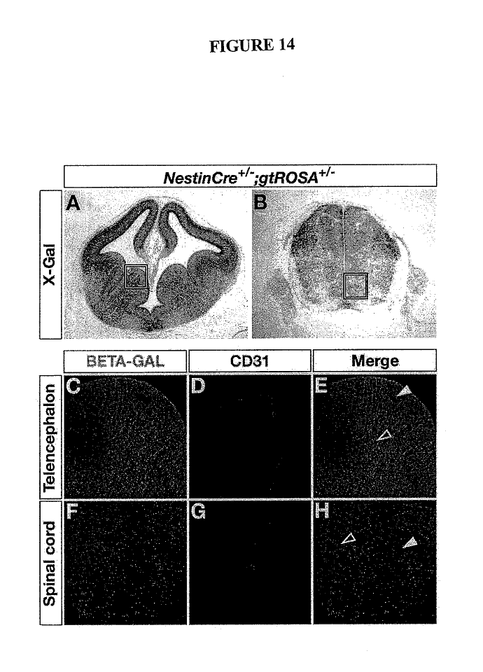

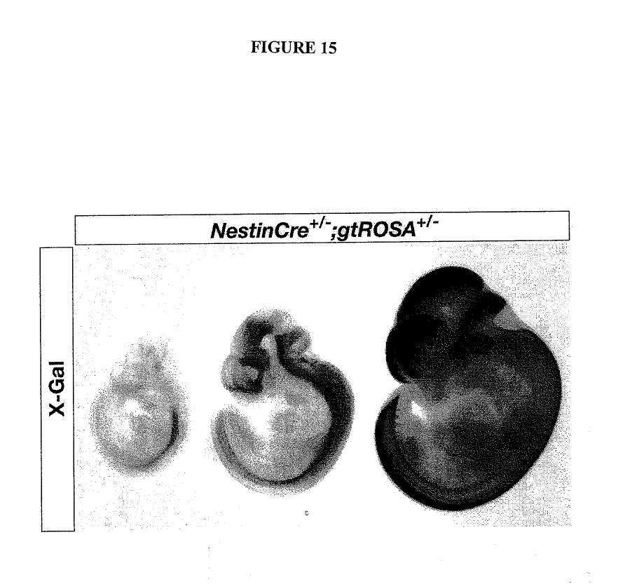

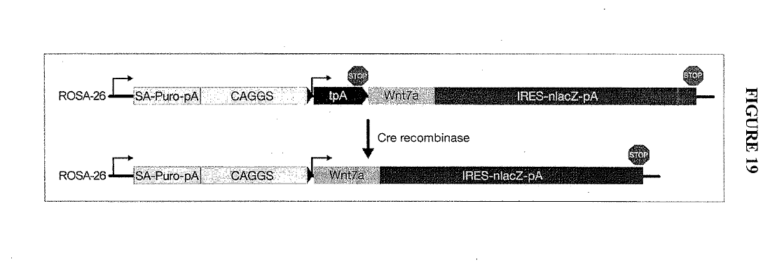

To confirm that the Wnt7a/b double mutant phenotype results from the loss of Wnt7a/b signaling within the developing neuroepithelium specifically, we used a NestinCre allele that drives expression of Cre recombinase predominantly in the neuroepithelium, excluding endothelial cells (FIG. 14; (11, 12)). Only partial recombination occurs by E9.5 and E10.5 in this model (FIG. 15; (13)), thus we predict a less severe phenotype than that of Sox2Cre; Wnt7a/b embryos. Nevertheless, we observe a CNS-specific hemorrhaging in the NestinCre; Wnt7a/b double mutants at E12.5 (FIG. 8Q, R). A similar molecular analysis to that reported earlier revealed the presence of endothelial cells in the PNVP and neuroepithelium (FIG. 19, T, FIG. 13, and data not shown). However, the number of endothelial cells and pericytes were reduced and the morphology of the vessels making up the INVP was abnormal.

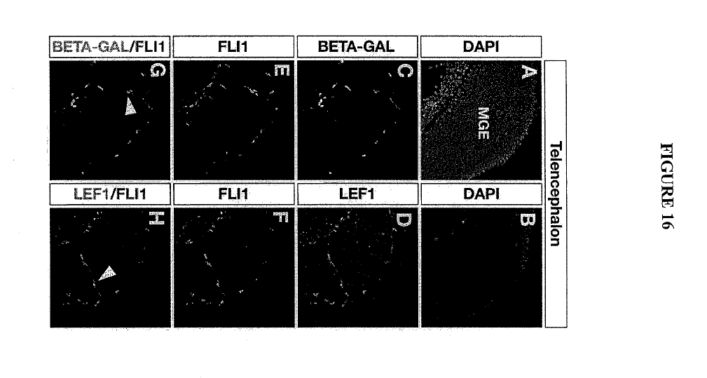

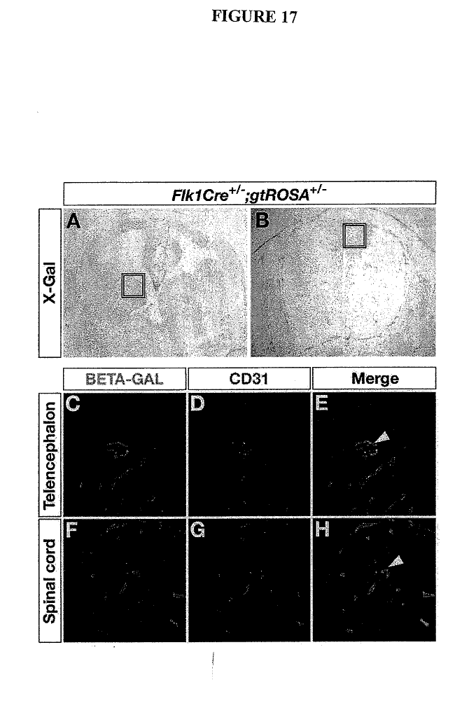

Next, we examined the underlying signaling pathway mediating Wnt7a/7b action and their cellular target. The BAT-Gal transgenic mouse contains a βCat-activated transgene which drives expression of an E. coli lacZ allele encoding nuclear β-galactosidase under the control of βCat/T-cell factor responsive elements (14). Consequently, BAT-Gal activity reports active canonical Wnt signaling (14). We observed β-galactosidase in a subset of FLI1-positive endothelial cells (15) in the PNVP and INVP at E10.5 (FIG. 9A, C, E, G, FIG. 16; (14)). Further, LEFT, a critical component and direct feed-forward target of canonical Wnt signaling, is expressed in endothelial cells of the PNVP and INVP (FIG. 9B, D, F, H, FIG. 16). Together, these data suggest that Wnt7a and Wnt7b may act directly on the endothelium via the canonical Wnt pathway. To test this possibility, we removed beta-catenin (βCat), an essential component of the canonical Wnt signaling transcriptional complex, in the endothelium combining a Flk1-Cre driver line (FIG. 17; (16)) and a conditional βCat allele (17).

Remarkably, removal of βCat in the endothelium results in a phenotype strikingly similar to that of the Wnt7a/b double mutant phenotype (FIG. 9I, J, M, N). As in Wnt7a/b double mutants, pericytes and endothelial cells were present in the PNVP at E12 (FIG. 9K, L, O, P, and data not shown). However, a more extensive loss of FLK1- and PDGFRβ-positive cells is evident in the neuroepithelium (FIG. 9K, L, O, P). When βCat activity was removed from the neuroepithelium using the NestinCre allele, we observed no hemorrhaging at E12.5 and the PNVP and INVP are indistinguishable from control embryos (FIG. 9Q-Y). Taken together, these results argue that Wnt ligands act directly on endothelial cells via the canonical pathway to regulate CNS vascularization. Importantly, in the Flk1 model, the vasculature outside the CNS forms. Thus canonical Wnt signaling plays an organ-specific role in vascularization. The more severe neural tube vascularization phenotype following Flk1-mediated βCat removal in all neural tube vascular progenitors is consistent with a more general role that extends to other Wnts in the regulation of CNS vascularization.

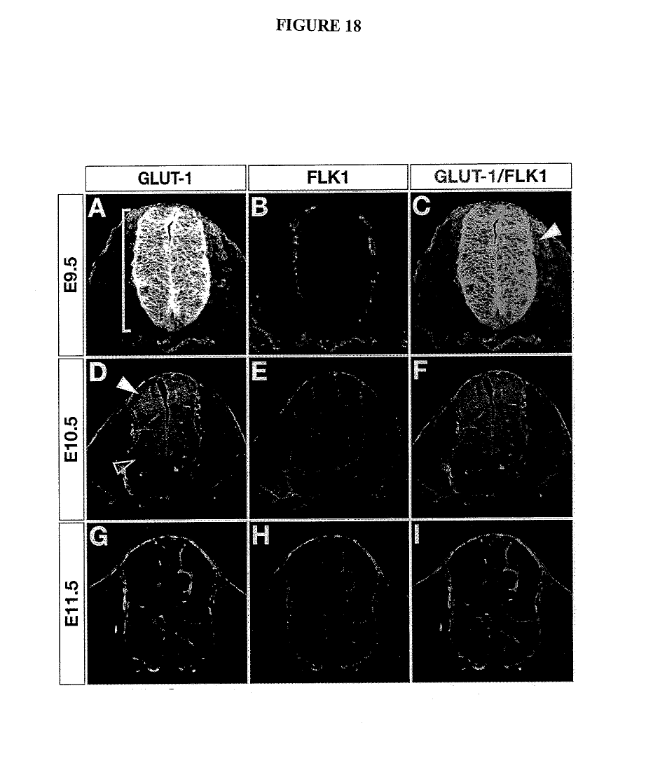

As mentioned earlier, the mature CNS endothelium exhibits a unique property referred to as the BBB for which GLUT-1 is an important indicator. Prior to CNS vascular invasion, GLUT-1 is present in the neuroepithelial cells, but as vascular invasion begins, GLUT-1 is downregulated in the neuroepithelium and upregulated in the vascular endothelium of the neural tube; high levels of GLUT-1 are maintained in CNS vasculature in the adult (FIG. 18 and data not shown).

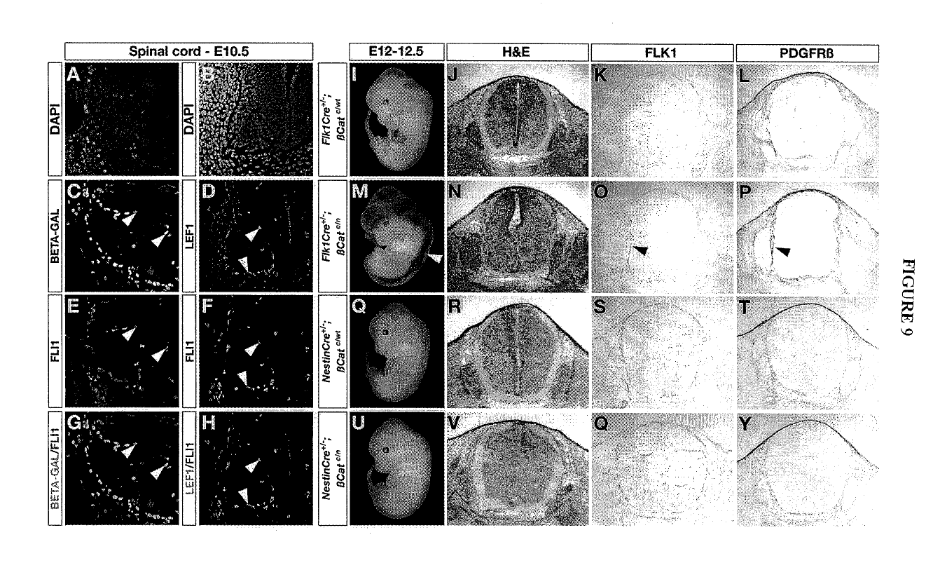

At E12.5, Wnt7a/b double mutants show reduced levels of GLUT-1 in the PNVP and INVP (FIG. 10A, E). Interestingly, weak GLUT-1 remains in the ventral neuroepithelium, where vascular invasion fails (FIG. 10A-H). In Flk1Cre; βCat mutant embryos, GLUT-1 is dramatically downregulated in the PNVP and GLUT-1 is maintained throughout the neuroepithelium (FIGS. 10I-P). In addition to the developing CNS vasculature, the developing limb endothelium also shows elevated GLUT-1 levels but these are not downreuglated in the Flk1Cre; βCat mutants. Taken together, these findings support a direct role for canonical Wnt signaling in organ-specific endothelial cell differentiation.

As shown in FIG. 10, canonical Wnt signaling involving Wnt7a and Wnt7b is necessary for proper differentiation of the CNS vasculature. Given that the vascular phenotype of the Wnt7a/b double mutant is less severe than the Flk1/Cre; βCat mutant embryos, it is apparent that other Wnt molecules (such as Wnt4) are associated with vasculogenesis and blood vessel differentiation. As such, the invention provides for the co-modulation of Wnt7a/b and one or more other Wnt ligands.

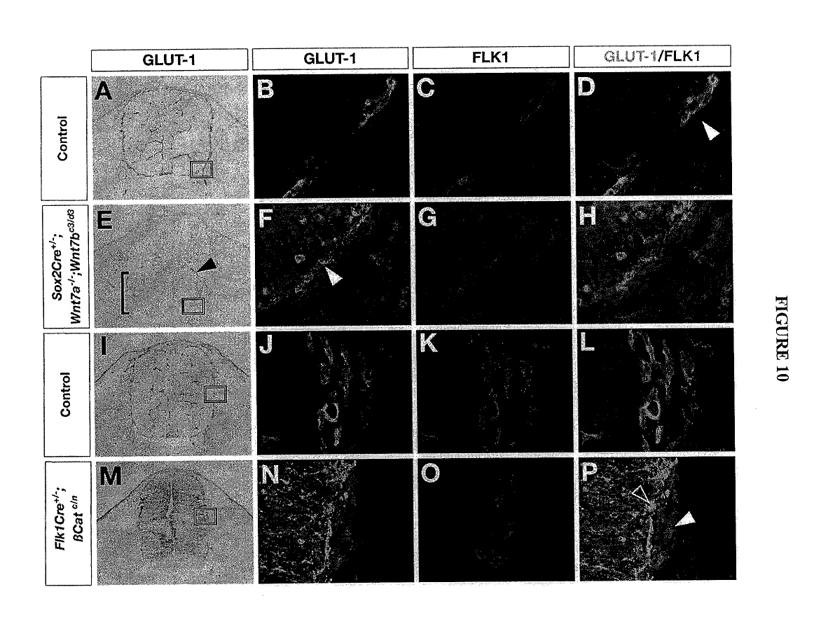

In order to test whether members of the Wnt7 sub-family of Wnt signals are sufficient to induce GLUT-1 in endothelial cells, we targeted the Rosa26 locus with a construct enabling Cre-mediated control of the expression of a bicistronic message encoding Wnt7a and lacZ (FIG. 19). The NestinCre allele, in addition to driving expression of Cre in the developing neuroepithelium, shows low-level sporadic recombination in scattered cells outside of the CNS at the forelimb level (FIG. 11A, E). Remarkably, we observed a strong upregulation of GLUT-1 in endothelial cells outside of the developing CNS at E10.5 and E11.5 in conjunction with ectopic Wnt7a expression driven by the NestinCre transgene (FIG. 11B-D, F-H).

Previous in vivo studies have suggested a role for Wnt7b in the development of the hyaloid and the lung vasculature via indirect mechanisms (18, 19), and have linked Wnt2 to the placental vasculature (20) and the putative Wnt receptor Fzd5 to yolk sac (21) vasculature. Additionally, in vitro studies suggest a role for Wnt signaling in proliferation, survival, and formation of capillary-like networks, suggesting angiogenic activity (22-26). Interestingly, βCat has previously been removed in the endothelium resulting in defective vascular patterning and increased vascular fragility, a phenotype attributed to the destabilization of adherens junctions rather than canonical Wnt signaling (27). The differences in phenotypes to those reported here may potentially be attributable to the use of different Cre driver lines, Tie2-Cre versus Flk1-Cre.

Our data provide in vivo evidence that Wnt7a and Wnt7b are critical regulators of organ-specific angiogenesis, acting directly on the CNS endothelium via the canonical pathway to promote both formation and differentiation of the CNS vasculature. Further, the data suggest this action may extend to other Wnts, particularly those present in dorsal regions of the neural tube. These findings may have important clinical ramifications. For example, local reductions in Wnt signaling levels could potentially lead to malformation of blood vessels as observed in the Wnt7a/b double mutants. In addition, the expression of Wnt7a/7b extends into the adult brain. If, BBB properties remain under Wnt control, altering Wnt7a/7b activity may be a fruitful strategy for delivery of pharmacological agents to the CNS. Further, there is a correlation between neo-angiogenesis and βCat accumulation in the endothelium of brain tumors, for example gliomas and human glioblastoma multiforme (28, 29). This raises the interesting possibility that canonical Wnt signaling could support disease, as it does development.

### Materials and Methods

### Animals and Genotyping

Animal care and research protocols were in accordance with institutional guidelines, and approved by the institutional committee on animal use. For staging of embryos the morning of vaginal plug was designated as embryonic day 0.5 (E0.5). Wnt7a (1), Wnt7b (2), Sox2Cre (3), Flk1Cre (4), NestinCre (5), and Beta-catenin (6) mice and embryos were genotyped by PCR using the following primers: Wnt7a-forward CTC TTC GGT GGT AGC TCT G, Wnt7a-reverse ACA CCC CAC ATT TAA ATT CAG G, Wnt7a-reverse-mutant TCA CGT CCT GCA CGA CGC, Wnt7b-null-forward GAG GAA GTC AGG CAG GTG TC, Wnt7b-null-reverse TAT CCC ACC GAT ACG CAA AC, Sox2Cre-forward CTC TAG AGC CTC TGC TAA CC, Sox2Cre-reverse CCT GGC GAT CCC TGA ACA TGT CC, Flk1Cre-forward TTG CTC TCA GAT GCG ACT TG, Flk1Cre-reverse TGC GAA CCT CAT CAC TCG TTG C, NestinCre-forward CGC TGG GTC ACT GTC GCC, NestinCre-reverse CCA TGA GTG AAC GAA CCT GG, Beta-catenin-forward-1 AAG GTA GAG TGA TGA AAG TTG TT, Beta-catenin-forward-2 TAC ACT ATT GAA TCA CAG GGA, and Beta-catenin-reverse CAC CAT GTC CTC TGT CTA TTC. B6.Cg-Tg(BAT-lacZ)3Picc/J mice (BATGAL) (7) were obtained from Jackson laboratories and genotyped as described in Jackson's genotyping protocol available online (http://www.jax.org). B6; 129-Gtrosa26tm1Sho (gtROSA) mice (8) were obtained from Jackson laboratories and genotyped as described in Jackson's Gtrosa genotyping protocol available online (http://www.jax.org). The generation of the conditional Wnt7b allele will be described elsewhere. Briefly, the conditional Wnt7b allele (Wnt7b^{c3}) has loxP sites flanking the essential exon 3 and phenocopies the null allele reported by Parr et al (2). The Wnt7b^{c3} allele was genotyped using the following primers: Wnt7b^{c3}-forward TGA CAG AGG ATG GGG AGA AG and Wnt7b^{c3}-reverse GGT CTT TCC AAG GGT GGT CT.

The R26-Wnt7aIRESLacZ allele was generated by first assembling a construct containing the splice acceptor from pBigT (9), followed by the puromycin resistance gene, the bovine growth hormone polyA sequence, the CAGGS enhancer/promoter/intron/SA cassette from pCAGGS (10), a floxed tpA (three SV40 polyA sequences from pBigT), a cassette containing IRESLacZ preceded by a nuclear localization signal, and finally the pCAGGS polyA sequence. Subsequently, Wnt7a was PCR amplified and subcloned in-between the floxed tpA and IRES2LacZ cassettes. Finally, the entire construct was released with Pad and AscI and subcloned into the Pad and AscI sites of pRosa26PAS (11). The construct was linearized and electroporated into F1 ES cells (12). Colonies were screened by PCR using the following primers: Rosa26-5armFlanking CCT AAA GAA GAG GCT GTG CTT TGG and Rosa26-SA CAT CAA GGA AAC CCT GGA CTA CTG. Positive colonies were expanded and confirmed by southern using both 5′ and 3′ probes. Two targeted clones were injected into host (C57BL/6J, Jackson Laboratories) blastocysts by the Genome Manipulation Facility, MCB, Harvard University. Mice and embryos were genotyped using the following primers: R26-Wnt7a-forward TAT ACG AAG TTA TTT TGT CGA CGC, and R26-Wnt7a-reverse ATT TGG GAG CCT TCT CCT AT.

### Histological Analysis

Embryos were fixed by immersion in 4% paraformaldehyde at 4° C. for 30 minutes to 4 hours (for immunohistochemistry) or 24 hours (for in situ hybridization). The embryos were rinsed thoroughly in PBS, incubated in 30% sucrose for at least 24 hours, embedded in OCT and frozen in a dry ice/ethanol bath. A cryostat was used to collect sections for subsequent histological analysis. Immunohistochemistry was performed on slide-mounted sections. Endogenous peroxidase activity was inactivated in sections that were to be processed with DAB using 0.3% H₂O₂ for 10-15 min. The sections were incubated with the primary antibody, 2% normal serum and 0.25% triton-X in PBS over night, and then incubated with an appropriate biotinylated secondary antibody, 2% normal serum and 0.25% triton-X in PBS. Finally the sections were processed using the ABC method (Vector Labs) with DAB as the final chromogen Immunofluorescent staining was performed essentially as previously described (13, 14). Confocal microscopy was performed on a Zeiss LSM510 confocal microscope.

Primary antibodies were used at the following concentrations: rat anti-CD31 (1:1000, BD Pharmingen), rabbit anti-FLK1 (1:1000, BD Pharmingen), goat anti-LEF1 (1:100, Santa Cruz), chicken anti-beta-gal (1:500, Abcam), rabbit anti-beta-gal (1:500, Cappel), rat anti-PDGFRβ (1:100, eBiosciences), rabbit anti-Glut1 (1:1000, Lab Vision), rabbit anti-Fli1 (1:500, Lab Vision). Secondary antibodies were acquired from DAKO, Molecular probes, and Jackson ImmunoResearch.

Whole-mount and section digoxigenin in situ hybridization was performed as described (15). X-gal histochemistry was carried out on slide-mounted sections. First, they were washed in 0.02% NP-40 and 2 mM MgCl₂ in PBS. They were then incubated in the staining solution (5 mM K₃Fe(CN)₆, 5 mM K₄Fe(CN)₆, 2 mM MgCl₂, 0.01% Deoxycholate, 0.02% NP-40, and 1 mg/ml X-gal) at 37° C. until the stain was clearly visible. The sections were then counterstained with Nuclear Fast Red Solution (Sigma-Aldrich).

### REFERENCES

A. M. Suburo, P. A. D'Amore, Handb Exp Pharmacol, 71 (2006).
B. Engelhardt, Cell Tissue Res 314, 119 (2003).
Miniño A M, Heron M P, Smith B L. Deaths: Preliminary Data for 2004. National vital statistics reports; vol 54 no 19. Hyattsville, Md.: National Center for Health Statistics, 2006.
C. L. Farrell, W. Risau, Microsc Res Tech 27, 495 (1994).
K. A. Hogan, C. A. Ambler, D. L. Chapman, V. L. Bautch, Development 131, 1503 (2004).
B. A. Parr, M. J. Shea, G. Vassileva, A. P. McMahon, Development 119, 247 (1993).
B. A. Parr, A. P. McMahon, Nature 395, 707 (1998).
B. A. Parr, V. A. Cornish, M. I. Cybulsky, A. P. McMahon, Dev Biol 237, 324 (2001).
S. Hayashi, P. Lewis, L. Pevny, A. P. McMahon, Mech Dev 119 Suppl 1, S97 (2002).
F. Tronche et al., Nat Genet. 23, 99 (1999).
D. Graus-Porta et al., Neuron 31, 367 (2001).
M. Backman et al., Dev Biol 279, 155 (2005).
S. Maretto et al., Proc Natl Acad Sci USA 100, 3299 (2003).
L. A. Brown et al., Mech Dev 90, 237 (2000).
T. Motoike, D. W. Markham, J. Rossant, T. N. Sato, Genesis 35, 153 (2003).
V. Brault et al., Development 128, 1253 (2001).
I. B. Lobov et al., Nature 437, 417 (2005).
W. Shu, Y. Q. Jiang, M. M. Lu, E. E. Morrisey, Development 129, 4831 (2002).
S. J. Monkley, S. J. Delaney, D. J. Pennisi, J. H. Christiansen, B. J. Wainwright, Development 122, 3343 (1996).
T. Ishikawa et al., Development 128, 25 (2001).
A. M. Goodwin, J. Kitajewski, P. A. D'Amore, Growth Factors 25, 25 (2007).
K. Venkiteswaran et al., Am J Physiol Cell Physiol 283, C811 (2002).
M. Wright, M. Aikawa, W. Szeto, J. Papkoff, Biochem Biophys Res Commun 263, 384 (1999).
T. N. Masckauchan at al., Mol Biol Cell 17, 5163 (2006).
T. N. Masckauchan, C. J. Shawber, Y. Funahashi, C. M. Li, J. Kitajewski, Angiogenesis 8, 43 (2005).
A. Cattelino et al., J Cell Biol 162, 1111 (2003).
H. Yano et al., Neural Res 22, 527 (2000).
H. Yano et al., Neural Res 22, 650 (2000).
B. A. Parr, V. A. Cornish, M. I. Cybulsky, A. P. McMahon, Dev Biol 237, 324 (2001).
X. Mao, Y. Fujiwara, S. Orkin, Proc Natl Acad Sci USA 96, 5037 (1999).
S. Srinivas at al., BMC Dev Biol 1, 4 (2001).
H. Niwa, K. Yarnamura, J. Miyazaki, Gene 108, 193 (1991).
J. Mao, J. Barrow, J. McMahon, J. Vaughan, A. P. McMahon, Nucleic Acids Res 33, c155 (2005).
K. Eggan et al., Proc Natl Acad Sci USA 98, 6209 (2001).
I. Jeong, A. P. McMahon, J Clin Invest 110, 591 (2002).
M. Wijgerde, J. McMahon, M. Rule, A. McMahon, Genes Dev 16, 2849 (2002).
D. Wilkinson, In Situ Hybridization: A Practical Approach (IRL Press at Oxford University Press, Oxford; New York, 1992), pp.

### GOVERNMENTAL SUPPORT

This invention was made with U.S. government support under DK054364 awarded by the National Institutes of Health. The U.S. government has certain rights in the invention.

### INCORPORATION BY REFERENCE

The contents of all cited references (including literature references, issued patents, published patent applications and GenBank Accession numbers as cited throughout this application) are hereby expressly incorporated by reference in their entireties.

### EQUIVALENTS

Those skilled in the art will recognize, or be able to ascertain using no more than routine experimentation, many equivalents of the specific embodiments of the invention described herein. Such equivalents are intended to be encompassed by the following claims.

1-36. (canceled)

37. A method for identifying an agent that modulates the activity of a Wnt ligand or a canonical Wnt signaling pathway component, comprising:
(i) contacting the Wnt ligand or the canonical Wnt signaling pathway component, or a portion thereof that is sufficient to promote canonical Wnt signaling, with a test agent; and
(ii) determining the level of activity from the Wnt ligand or the canonical Wnt signaling pathway component or portion thereof in the presence of the test agent relative to the absence of the test agent, wherein a change in activity indicates that the test agent is an agent that modulates the activity of the Wnt ligand or the canonical Wnt signaling pathway component.

38. The method of claim 37, wherein the test agent stimulates or inhibits the level of activity of the Wnt ligand or the canonical Wnt signaling pathway component or portion thereof relative to the level of activity in the absence of the test agent.

39. The method of claim 37, wherein the Wnt ligand is human Wnt7a, human Wnt7b, mouse Wnt7a or mouse Wnt7b.

40. The method of claim 37, wherein the test agent is a small molecule.

41. An animal model for vascular disorders of the CNS and disorders of the blood-brain barrier, consisting of an animal having a mutation in one or more genes encoding a Wnt ligand or a canonical Wnt signaling pathway component, wherein the mutation prevents the canonical Wnt signaling pathway from functioning normally and causes a hemorrhaging phenotype.

42. The animal model of claim 41, wherein the mutation is a Wnt7a/b double mutant.

43. A method for treating a disease or disorder of the CNS or the blood-brain barrier in a subject, comprising administering to the subject an effective amount of an agent that modulates the activity of a Wnt ligand or the activity of a canonical Wnt signaling pathway component.

44. The method of claim 43, wherein the agent modulates the activity of human Wnt7a, human Wnt7b, mouse Wnt7a or mouse Wnt7b.

45. The method of claim 44, wherein the method comprises modulating blood-brain barrier (BBB) permeability in the subject.

46. The method of claim 45, wherein said agent is a Wnt7a ligand or a Wnt7b ligand.

47. The method of claim 45, wherein the agent increases or decreases BBB permeability.

48. The method of claim 45, wherein the modulating is reversible.

49. The method of claim 44, wherein the agent is selected from the group consisting of an inorganic molecule, peptide, peptide mimetic, antibody, liposome, small interfering RNA, antisense, aptamer, external guide sequence, and combinations thereof.

50. The method of claim 44, wherein the agent is a secreted Wnt 7a/7b signaling inhibitor.

51. The method of claim 44, further comprising administering a second agent.

52. The method of claim 44, wherein the subject has a CNS tumor.

53. The method of claim 52, further comprising administering a radiotherapeutic or chemotherapeutic agent.

54. The method of claim 52, wherein the CNS tumor is a neuronal tumor, a glial tumor, a menigneal tumor, a pituitary tumor, a pineal gland tumor, or a lymphatic tumor.

55. The method of claim 44, wherein said method comprises inducing angiogenesis in the central nervous system of a mammalian subject.

56. The method of claim 55, wherein the mammalian subject has suffered a stroke, has been exposed to perinatal hypoxia, has been exposed to perinatal ischemia, or has suffered a spinal cord injury.


SEQUENCE LISTING (28 sequences)

SEQ ID NO: 1 - DNA, 1732 bp, Homo sapiens
gaggggcgggggctggaggcagcagcgcccccgcactccccgcgtctcgcacacttgcaccggtcgctcgcgcgcagcccggcgtcgccccacgccgcgctcgctcctccctccctcctcccgctccgtggctcccgtgctcctggcgaggctcaggcgcggagcgcgcggacgggcgcaccgacagacggccccggggacgcctcggctcgcgcctcccgggcgggctatgttgattgccccgccggggccggcccgcgggatcagcacagcccggcccgcggccccggcggccaatcgggactatgaaccggaaagcgcggcgctgcctgggccacctctttctcagcctgggcatggtctacctccggatcggtggcttctcctcagtggtagctctgggcgcaagcatcatctgtaacaagatcccaggcctggctcccagacagcgggcgatctgccagagccggcccgacgccatcatcgtcataggagaaggctcacaaatgggcctggacgagtgtcagtttcagttccgcaatggccgctggaactgctctgcactgggagagcgcaccgtcttcgggaaggagctcaaagtggggagccgggaggctgcgttcacctacgccatcattgccgccggcgtggcccacgccatcacagctgcctgtacccagggcaacctgagcgactgtggctgcgacaaagagaagcaaggccagtaccaccgggacgagggctggaagtggggtggctgctctgccgacatccgctacggcatcggcttcgccaaggtctttgtggatgcccgggagatcaagcagaatgcccggactctcatgaacttgcacaacaacgaggcaggccgaaagatcctggaggagaacatgaagctggaatgtaagtgccacggcgtgtcaggctcgtgcaccaccaagacgtgctggaccacactgccacagtttcgggagctgggctacgtgctcaaggacaagtacaacgaggccgttcacgtggagcctgtgcgtgccagccgcaacaagcggcccaccttcctgaagatcaagaagccactgtcgtaccgcaagcccatggacacggacctggtgtacatcgagaagtcgcccaactactgcgaggaggacccggtgaccggcagtgtgggcacccagggccgcgcctgcaacaagacggctccccaggccagcggctgtgacctcatgtgctgtgggcgtggctacaacacccaccagtacgcccgcgtgtggcagtgcaactgtaagttccactggtgctgctatgtcaagtgcaacacgtgcagcgagcgcacggagatgtacacgtgcaagtgagccccgtgtgcacaccaccctcccgctgcaagtcagattgctgggaggactggaccgtttccaagctgcgggctccctggcaggatgctgagcttgtcttttctgctgaggagggtacttttcctgggtttcctgcaggcatccgtgggggaaaaaaaatctctcagagccctcaactattctgttccacacccaatgctgctccaccctcccccagacacagcccaggtccctccgcggctggagcgaagccttctgcagcaggaactctggacccctgggcctcatcacagcaatatttaacaatttattctgataaaaataatattaatttatttaattaaaaagaattcttccacaaaaaaaaaaaaaaaaaa

SEQ ID NO: 2 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 3 - DNA, 3176 bp, Mus sp.
gcagtccccgcgcctcaaacacttgccgcgatcgctggcgcgcagcggcgccccttgttgcgcttgttctcccctcctctggctccgcggctcccgcgctctgggacagtctccagtgcctagcgcggaccgacgcaccgacggaccgcccagggagcctcggcccgcgccccctgcgcaggctatgtggattgccccgccgggcccggctggcgggatcagcacagcccggcccgtggcacccgccaccagcggggactatgacccggaaagcgcggcgctgcctgggccacctctttctcagcctgggcatagtctacctccggatcggtggcttctcttcggtggtagctctgggtgcgagcatcatctgtaacaagatcccaggcctggctcccagacagcgggcaatctgccagagccggccggacgccatcatcgtcataggagaaggctcccaaatgggcctggacgagtgtcagtttcagttccgaaatggccgttggaactgctcagcgctgggagagcgtactgtcttcgggaaggagctcaaagtggggagtcgggaggctgccttcacctatgcgattatcgctgcgggcgtggcccatgccatcactgctgcctgcacccagggcaacctgagcgactgtggctgcgacaaggagaagcaaggccagtaccaccgggacgagggctggaagtggggtggctgctctgccgacatccgctacggcatcggcttcgccaaggtcttcgtggatgcccgggagatcaagcagaatgcccggacgctcatgaacttacacaataacgaggcgggtcggaagatcctggaggagaacatgaagctggagtgtaagtgccatggtgtgtcaggctcctgtaccactaagacgtgctggaccacactgccacagttccgagagctaggctacgtgctcaaggacaaatacaacgaggccgtccacgtggagcctgtgcgtgccagtcgaaacaagcggcccacctttctgaagatcaagaagcccctgtcctaccgcaagcccatggacactgacctggtgtatatcgagaagtcacccaattactgtgaagaggacccagtgacaggcagcgtgggtacccagggccgagcctgcaataagacagcccctcaggccagtggctgtgacctcatgtgctgtggccgtggctacaacacacaccagtacgcccgggtgtggcagtgcaactgcaaattccactggtgctgctacgtcaagtgtaacacgtgcagcgagcgcacggagatgtatacgtgcaagtgaatgcggtcacaggtcagatcacaggcaggatacagtttccctgcaggccactgcctggatgctcacagggaaagaaccacagaagcactgtccttgtcttttctgctgaggggggaggggtattctgggtttcctgcagactcccgtgggaagcatctctcagaggcccgcccattcttctccacatggatgctgctcagccaccctcccccagacaccgcccgagcctctccagggctggaacaaagttttctacggcaggagctctggagcctcgggcctcgtcatagcaatatttaacagtttattctgatatgagataatattaatttatttaattaaagagaattcttccacttcgtcgggatccgtcttctgcaatcaaagtggactgcttgaggtcctggtgggatgacttgctaggactgggagctgagaacagctgtacataattattctttatgcagatgtttctactagttgatttcacaagtacccttctgcagcgctaggtgttaagtacaaagagaagacggtctttatacacatatagatatatatatgcatacacatttgtaactttgttttgttttgtttttgctgtttgctgctacctatccagactctaagctggtccagatctggaattgtttttctccaggacgtgctcctatccttttgccctttacagttcaaacctctccgttagaaaagttccattgggaatggcgtgtgtgtgatggggacgaggatcacaaattcccagcagtttccatcctgaaacgtgaaccactggataagaggctttctaagagactatttttctatggatattttatttatatggagtctgcctgcggtgccccatggcccatgcctcttcttaacactggtactcactcaggggcagaaggacaaggccaggtgtgtgggcaggtcccccggggaccctcacacagctggagcctggagttctatttgccaagggggccatagcagttaccagatgcctgggttgggtatcttctgtgttaaacaagagggaaccatcccctggctttagcctgctaagctcagggcttggaatggggtcactggatggttatcttgggagatgacctctggatgagcctcagcggtgggtcagtcagtgtctcacacactttgagaagcatgggacctggcattcatcatcaggcagaggccagctcagggatgccgctatcccatcaggacagcccaggcactgcctctaggtgaggtgtagtcctaagagaaggggtcaaggagggggaaggaggaagccaaggagtgttggccatcctcagtgaaagcgatgggagcgttctctcagcagcagagacacagctgtacctgtatctctccaatgggaaacccctccagaaggctggggatattttttatgtgtttccacatgcatttccacctgtgtgcatgtaagcacatgcgcacactcctgtgccagcactctgcggcacctccagggtgctcacgggtacatgtgcttacatgtatctctctgtgcttgggagatcagaccatgtgcatggagctgtatgcctgagcacttgtggtctcaggggttatttccaggtatctgcatttgtgggtggggtgcaaggtagacagcagggaactgatttgattgtgttgagccacagtgagactgcaactctgaactctgtctccacagctgctggtgaaactcagatgcctgtgagacaacagccctgagcctcatggcccacatgctgggagcccctcagtgtctaggtcatgtccagtcccccacctgggttacatcacgaccaataaacatggctgtatggctgatttcttcccttg

SEQ ID NO: 4 - protein, 349 aa, Mus sp.
MTRKARRCLGHLFLSLGIVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 5 - DNA, 2250 bp, Homo sapiens
gagtctgcccgcagccccctggcccctgcccggccctgcgtgcccgcgcgtccctccggccgcgctgtctatggcgcagcccccctccctggatcatgcacagaaactttcgcaagtggattttctacgtgtttctctgctttggcgtcctgtacgtgaagctcggagcactgtcatccgtggtggccctgggagccaacatcatctgcaacaagattcctggcctagccccgcggcagcgtgccatctgccagagtcggcccgatgccatcattgtgattggggagggggcgcagatgggcatcaacgagtgccagtaccagttccgcttcggacgctggaactgctctgccctcggcgagaagaccgtcttcgggcaagagctccgagtagggagccgtgaggctgccttcacgtacgccatcaccgcggctggcgtggcgcacgccgtcaccgctgcctgcagccaagggaacctgagcaactgcggctgcgaccgcgagaagcagggctactacaaccaagccgagggctggaagtggggcggctgctcggccgacgtgcgttacggcatcgacttctcccggcgcttcgtggacgctcgggagatcaagaagaacgcgcggcgcctcatgaacctgcataacaatgaggccggcaggaaggttctagaggaccggatgcagctggagtgcaagtgccacggcgtgtctggctcctgcaccaccaaaacctgctggaccacgctgcccaagttccgagaggtgggccacctgctgaaggagaagtacaacgcggccgtgcaggtggaggtggtgcgggccagccgtctgcggcagcccaccttcctgcgcatcaaacagctgcgcagctatcagaagcccatggagacagacctggtgtacattgagaagtcgcccaactactgcgaggaggacgcggccacgggcagcgtgggcacgcagggccgtctctgcaaccgcacgtcgcccggcgcggacggctgtgacaccatgtgctgcggccgaggctacaacacccaccagtacaccaaggtgtggcagtgcaactgcaaattccactggtgctgcttcgtcaagtgcaacacctgcagcgagcgcaccgaggtcttcacctgcaagtgaggccaggcccggaggcggccgcgggcaccctggaacccggcggcattttgcacatccactcctcaccttccctgccttggtgctgccagcagcagacatagacgggtgcagaagcggggagctccaggtgcaggagggcaccggccggggcccacgccctctgcccgcctccctggggctccttcctgccacctcctcccatcacctcctgcggcagaacagcacccgtgacccacccagagagcaaggccaggggtcttggtgctccccgacggggcccggcaagttctctttcttctctctgggaaaatgaacgtccaggacacacctgtatcccagagagcaaagtgatgaggagactgagcgtccccagccccacctggcggcatggacacagaaaagctacgccggctggcctctccagaccagttcccaggctgggtctgccgctgggccctggggcggtggggacagatgttgacacaaattatttatgttttcttagtatcagaagaggattctcggcactaacacatagccagtcctaactccgtactctgtgtcagcccatcccctagacaccctctgtttcctttcccggccccacctggccggccctctgcccctgcagagctgaggcagcctggggttgatggggaccacgcggtgcctgcaggtcctagaagtgagctgggcaggggctcttcagaccacacagccctgaccgggccttggaggagagccatggacaggctcctccatgccgtctttccttcttttgaaaatcctatcaatggctgggcgcggtggctcacacctgtaatcccagcactttgggagaccgaggcaggtggatcacctgaggtcaggagttcgagaccagcctggccaacgtggtgaaaccctgtctctactaaaaatacaaaaattagctgggcgtggtggcgtgcacctgtaatcccagctactcaggaggctgagacaggacacttgcttgaacccgggaggtggaggttgcaatgagccaagattgtgccactgtattccaacttgggtgacagagcacgactctgtctcaaaaaaaaaaaaaaaaaaaaaaaaaaaa

SEQ ID NO: 6 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 7 - DNA, 3154 bp, Mus sp.
cgcccgcctcccgagccgaagcgccggctgagcgtggtcctaccgcagctccctggctcctgcccggcccctgcccacccgcgcgtcccctccggccgcagctgtctatggcgcagcccccctccctggatcatgcacagaaactttcgaaagtggatcttttacgtgtttctctgctttggcgtcctctacgtgaagctcggagcattgtcatccgtggtggccctggtagccaacatcatctgcaacaagattcctggcctggccccacggcagcgtgccatctgccagagccgacccgatgccatcattgtgatcggggagggggcgcagatgggcatcgacgagtgccagcaccagttccgattcggccgctggaactgctccgccctgggcgagaagaccgtcttcgggcaagaactccgagtagggagtcgagaggctgccttcacctatgccatcacggcggcgggcgtggcgcatgctgtcaccgctgcctgcagccagggcaatctgagcaattgtggctgtgaccgggagaagcaaggctactacaaccaggcggaaggctggaagtgggggggctgctcagcggacgtccgctacggcatcgacttttctcgtcgctttgtggatgcccgtgagatcaaaaagaacgccaggcgcctcatgaaccttcacaacaatgaggcgggcagaaaggttctggaggaccgcatgaagctggaatgtaagtgtcacggtgtgtcaggctcctgtaccaccaaaacttgctggaccacgctacctaagttccgcgaggtgggccacctgctcaaggagaagtacaacgcagcggtgcaggtggaggtggtgcgagccagccgcctgcgccagcccaccttcctgcgcatcaagcagctacgcagctaccagaagcctatggagacggacctggtgtacatcgagaagtcgcccaactactgcgaggaggacgcggccacgggcagcgtgggcacgcagggccgtctgtgcaaccgcacctcgccgggggccgacggctgtgacaccatgtgctgcggccgcggctacaacacgcaccagtacaccaaggtgtggcagtgtaactgcaaattccactggtgttgcttcgtcaagtgcaacacgtgcagcgagcgcaccgaggtcttcacctgcaagtgaggctcccgcgcaggcgcgctcggcccctgccgaccctgcggccctcgccattattttgcacatccttctttgcttctggagctgccagctgcaggcacaggagggtggggatagaggtggggagctcgagatactccaggctccttcctactcgctctgtccccgcccagcatccaaggtcaacgcaatggtggtctggtacccaatggagacaaatccctttacttctctttgggaaagtgaaccacaaagggaccatgagactctgagggtcacctccctgcctgtgactggacacagaaaggccacacccaccagtcacactcaaaacggtttcctgggctgtttcctgccggccctgggcagtgtggatggatgttgacaaaattatttatgttttcttagcatcagatgaggactcagtactaacgactgggtagccagacctaaccctatttgaggacacccttccctcactcctcccggcccctccctgcagggtcctctgctccttgcagaactcgaggatgtcagaattggcacggaagctggctggtggggggactccttatcagcaccttgggaggggcttggtggccctacaaggcctgagatggccgcagaggacagccaatcttccattccatttggagactgtcatgcaaatcaaatgtcccttgtgtcaggctccaggcatgcctcgtcctctccctggtccttcaccctcccagcctgctgccaacctccacctccagtttacaaattctcttctcctctggagccaacctgacacccaggactgccccacaggttcaggagaggtcagggacagttgccccacatgacagatggacagagggcaatctgaagatttactggagaccccacggctctgtgaaataaatatactgacacagccccatccagcccaactctggaagttgccagggtgatgggaggctgcacccccttttcagtaccttgggttttgtccttcttctgtgatcctgatgccagagaactgacatccagaatttagggatgtattggtcaggccccctgcctagtgtccactgatacctgcttcagggtccttatattatgaggacatgggaccctcaaacaggggtccgtgggaagcttaatgtcccatttcctcaggcccttccagatggggacagaagaactcaggcctgggcatatcccaccctttcctccacaacacatggcagggtaagaaactgccagggctgataatacaactgcccacagcctaccccacactaaggtgtttcatagcagaagtccatggaaatgtggggtttggtggccaccaagccaggtggcctggacattgacctggggaaggtgacccttgtttgcccttgccttgcatccagctgtgtgtccctatcatgtcaggatgttccaagcctctgggccactggaaatgtcccaccctgatcctggccccatctcctcaccccaagtcctgggatacccacgtccgtcgcccagtgtcccctgtgaggagcctggttaacttatattgttatatagcgtcccctgtctgtcatgtctcttaagttattgtgacctacactgggtaccggaggggatgggggatggcttcagctgctgtcccccaagccaggctcctccttctgcttgaaacagaccctcgggggcccctgatgccaccgaggcaattcgcactgtccctgggctgccaggcacctgcgcctgcactcggtcagccgcagaccttgccttgggggagagaggtggttagtggacccaggcagggcactggctgtcccaatgctgtgtgctggggtggaggtggccgggcaccacatgtccttgaagtgccctacttctgatgggctgtgttcctgcctcctctggaggggagcacttagccccaataaaagctggaatcagaaaaaaaaaaaaaaaaaaaaaaaaaaaa

SEQ ID NO: 8 - protein, 349 aa, Mus sp.
MHRNFRKWIFYVFLCFGVLYVKLGALSSVVALVANIICNKIPGLAPRQRAICQSRPDAIIVIGEGAQMGIDECQHQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMKLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 9 - DNA, 19 bp, Artificial Sequence
  source: /note=“Description of Artificial Sequence Synthetic primer”
ctcttcggtggtagctctg

SEQ ID NO: 10 - DNA, 22 bp, Artificial Sequence
  source: /note=“Description of Artificial Sequence Synthetic primer”
acaccccacatttaaattcagg

SEQ ID NO: 11 - DNA, 18 bp, Artificial Sequence
  source: /note=“Description of Artificial Sequence Synthetic primer”
tcacgtcctgcacgacgc

SEQ ID NO: 12 - DNA, 20 bp, Artificial Sequence
  source: /note=“Description of Artificial Sequence Synthetic primer”
gaggaagtcaggcaggtgtc

SEQ ID NO: 13 - DNA, 20 bp, Artificial Sequence
  source: /note=“Description of Artificial Sequence Synthetic primer”
tatcccaccgatacgcaaac

SEQ ID NO: 14 - DNA, 20 bp, Artificial Sequence
  source: /note=“Description of Artificial Sequence Synthetic primer”
ctctagagcctctgctaacc

SEQ ID NO: 15 - DNA, 23 bp, Artificial Sequence
  source: /note=“Description of Artificial Sequence Synthetic primer”
cctggcgatccctgaacatgtcc

SEQ ID NO: 16 - DNA, 20 bp, Artificial Sequence
  source: /note=“Description of Artificial Sequence Synthetic primer”
ttgctctcagatgcgacttg

SEQ ID NO: 17 - DNA, 22 bp, Artificial Sequence
  source: /note=“Description of Artificial Sequence Synthetic primer”
tgcgaacctcatcactcgttgc

SEQ ID NO: 18 - DNA, 18 bp, Artificial Sequence
  source: /note=“Description of Artificial Sequence Synthetic primer”
cgctgggtcactgtcgcc

SEQ ID NO: 19 - DNA, 20 bp, Artificial Sequence
  source: /note=“Description of Artificial Sequence Synthetic primer”
ccatgagtgaacgaacctgg

SEQ ID NO: 20 - DNA, 23 bp, Artificial Sequence
  source: /note=“Description of Artificial Sequence Synthetic primer”
aaggtagagtgatgaaagttgtt

SEQ ID NO: 21 - DNA, 21 bp, Artificial Sequence
  source: /note=“Description of Artificial Sequence Synthetic primer”
tacactattgaatcacaggga

SEQ ID NO: 22 - DNA, 21 bp, Artificial Sequence
  source: /note=“Description of Artificial Sequence Synthetic primer”
caccatgtcctctgtctattc

SEQ ID NO: 23 - DNA, 20 bp, Artificial Sequence
  source: /note=“Description of Artificial Sequence Synthetic primer”
tgacagaggatggggagaag

SEQ ID NO: 24 - DNA, 20 bp, Artificial Sequence
  source: /note=“Description of Artificial Sequence Synthetic primer”
ggtctttccaagggtggtct

SEQ ID NO: 25 - DNA, 24 bp, Artificial Sequence
  source: /note=“Description of Artificial Sequence Synthetic primer”
cctaaagaagaggctgtgctttgg

SEQ ID NO: 26 - DNA, 24 bp, Artificial Sequence
  source: /note=“Description of Artificial Sequence Synthetic primer”
catcaaggaaaccctggactactg

SEQ ID NO: 27 - DNA, 24 bp, Artificial Sequence
  source: /note=“Description of Artificial Sequence Synthetic primer”
tatacgaagttattttgtcgacgc

SEQ ID NO: 28 - DNA, 20 bp, Artificial Sequence
  source: /note=“Description of Artificial Sequence Synthetic primer”
atttgggagccttctcctat